SEQUENCE LISTING (4 sequences)

SEQ ID NO: 1 - protein, 622 aa, Homo sapiens
MKENVASATVFTLLLFLNTCLLNGQLPPGKPEIFKCRSPNKETFTCWWRPGTDGGLPTNYSLTYHREGETLMHECPDYITGGPNSCHFGKQYTSMWRTYIMMVNATNQMGSSFSDELYVDVTYIVQPDPPLELAVEVKQPEDRKPYLWIKWSPPTLIDLKTGWFTLLYEIRLKPEKAAEWEIHFAGQQTEFKILSLHPGQKYLVQVRCKPDHGYWSAWSPATFIQIPSDFTMNDTTVWISVAVLSAVICLIIVWAVALKGYSMVTCIFPPVPGPKIKGFDAHLLEKGKSEELLSALGCQDFPPTSDYEDLLVEYLEVDDSEDQHLMSVHSKEHPSQGMKPTYLDPDTDSGRGSCDSPSLLSEKCEEPQANPSTFYDPEVIEKPENPETTHTWDPQCISMEGKIPYFHAGGSKCSTWPLPQPSQHNPRSSYHNITDVCELAVGPAGAPATLLNEAGKDALKSSQTIKSREEGKATQQREVESFHSETDQDTPWLLPQEKTPFGSAKPLDYVEIHKVNKDGALSLLPKQRENSGKPKKPGTPENNKEYAKVSGVMDNNILVLVPDPHAKNVACFEESAKEAPPSLEQNQAEKALANFTATSSKCRLQLGGLDYLDPACFTHSFH

SEQ ID NO: 2 - protein, 1205 aa, Homo sapiens
MNGYAEFPPSPSNPTKEPVEPQPSQVPLQEDVDMSSGSSGHETNENCSTGRDSQGSDCDDSGKELGMLVEPPDARQSPDTFSLMMAKSEHNPSTSGCSSDQSSKVDTHKELIKTLKELKVHLPADKKAKGKASTLATLKYALRSVKQVKANEEYYQLLMSSEGHPCGADVPSYTVEEMESVTSEHIVKNADMFAVAVSLVSGKILYISDQVASIFHCKRDAFSDAKFVEFLAPHDVGVFHSFTSPYKLPLWSMCSGADSFTQECMEEKSFFCRVSVRKSHENEIRYHPFRMTPYLVKVRDQQGAESQLCCLLLAERVHSGYEAPRIPPEKRIFTTTHTPNCLFQDVDERAVPLLGYLPQDLIETPVLVQLHPSDRPLMLAIHKKILQSGGQPFDYSPIRFRARNGEYITLDTSWSSFINPWSRKISFIIGRHKVRVGPLNEDVFAAHPCTEEKALHPSIQELTEQIHRLLLQPVPHSGSSGYGSLGSNGSHEHLMSQTSSSDSNGHEDSRRRRAEICKNGNKTKNRSHYSHESGEQKKKSVTEMQTNPPAEKKAVPAMEKDSLGVSFPEELACKNQPTCSYQQISCLDSVIRYLESCNEAATLKRKCEFPANVPALRSSDKRKATVSPGPHAGEAEPPSRVNSRTGVGTHLTSLALPGKAESVASLTSQCSYSSTIVHVGDKKPQPELEMVEDAASGPESLDCLAGPALACGLSQEKEPFKKLGLTKEVLAAHTQKEEQSFLQKFKEIRKLSIFQSHCHYYLQERSKGQPSERTAPGLRNTSGIDSPWKKTGKNRKLKSKRVKPRDSSESTGSGGPVSARPPLVGLNATAWSPSDTSQSSCPAVPFPAPVPAAYSLPVFPAPGTVAAPPAPPHASFTVPAVPVDLQHQFAVQPPPFPAPLAPVMAFMLPSYSFPSGTPNLPQAFFPSQPQFPSHPTLTSEMASASQPEFPEGGTGAMGTTGATETAAVGADCKPGTSRDQQPKAPLTRDEPSDTQNSDALSTSSGLLNLLLNEDLCSASGSAASESLGSGSLGCDASPSGAGSSDTSHTSKYFGSIDSSENNHKAKMNTGMEESEHFIKCVLQDPIWLLMADADSSVMMTYQLPSRNLEAVLKEDREKLKLLQKLQPRFTESQKQELREVHQWMQTGGLPAAIDVAECVYCENKEKGNICIPYEEDIPSLGLSEVSDTKEDENGSPLNHRIEEQT

SEQ ID NO: 3 - protein, 376 aa, Homo sapiens
MKENVASATVFTLLLFLNTCLLNGQLPPGKPEIFKCRSPNKETFTCWWRPGTDGGLPTNYSLTYHREGETLMHECPDYITGGPNSCHFGKQYTSMWRTYIMMVNATNQMGSSFSDELYVDVTYIVQPDPPLELAVEVKQPEDRKPYLWIKWSPPTLIDLKTGWFTLLYEIRLKPEKAAEWEIHFAGQQTEFKILSLHPGQKYLVQVRCKPDHGYWSAWSPATFIQIPSDFTMNDTTVWISVAVLSAVICLIIVWAVALKGYSMVTCIFPPVPGPKIKGFDAHLLEKGKSEELLSALGCQDFPPTSDYEDLLVEYLEVDDSEDQHLMSVHSKEHPSQGDPLMLGASHYKNLKSYRPRKISSQGRLAVFTKATLTTVQ

SEQ ID NO: 4 - protein, 1163 aa, synthetic construct
  REGION 1..1163: note = Description of Artificial Sequence: Synthetic polypeptide
MAGWIQAQQLQGDALRQMQVLYGQHFPIEVRHYLAQWIESQPWDAIDLDNPQDRAQATQLLEGLVQELQKKAEHQVGEDGFLLKIKLGHYATQLQKTYDRCPLELVRCIRHILYNEQRLVREANNCSSPAGILVDAMSQKHLQINQTFEELRLVTQDTENELKKLQQTQEYFIIQYQESLRIQAQFAQLAQLSPQERLSRETALQQKQVSLEAWLQREAQTLQQYRVELAEKHQKTLQLLRKQQTIILDDELIQWKRRQQLAGNGGPPEGSLDVLQSWCEKLAEIIWQNRQQIRRAEHLCQQLPIPGPVEEMLAEVNATITDIISALVTSTFIIEKQPPQVLKTQTKFAATVRLLVGGKLNVHMNPPQVKATIISEQQAKSLLKNENTRNECSGEILNNCCVMEYHQATGTLSAHFRNMSLKRIKRADRRGAESVTEEKFTVLFESQFSVGSNELVFQVKTLSLPWVIVHGSQDHNATATVLWDNAFAEPGRVPFAVPDKVLWPQLCEALNMKFKAEVQSNRGLTKENLVFLAQKLFNNSSSHLEDYSGLSVSWSQFNRENLPGWNYTFWQWFDGVMEVLKKHHKPHWNDGAILGFVNKQQAHDLLINKPDGTFLLRFSDSEIGGITIAWKFDSPERNLWNLKPFTTRDFSIRSLADRLGDLSYLIYVFPDRPKDEVFSKYYTPVLAKAVDGYVKPQIKQWPEFVNASADAGGSSATYMDQAPSPAVCPQAPYNMYPQNPDHVLDQDGEFDLDETMDVARHVEELLRRPMDSLDSRLSPPAGLFTSARGSLSLDSQRKLQFYEDRHQLPAPKWAELANLINNCMDYEPDFRPSFRAIIRDLNSLFTPDYELLTENDMLPNMRIGALGFSGAFEDRDPTQFEERHLKFLQQLGKGNFGSVEMCRYDPLQDNTGEWAVKKLQHSTEEHLRDFEREIEILKSLQHDNIVKYKGVCYSAGRRNLKLIMEYLPYGSLRDYLQKHKERIDHIKLLQYTSQICKGMEYLGTKRYIHRDLATRNILVENENRVKIGDFGLTKVLPQDKEYYKVKEPGESPIFWYAPESLTESKFSVASDVWSFGWLYELFTYIEKSKSPPAEFMRMIGNDKQGQMIVFHLIELLKNNGRLPRPDGCPDEIYMIMTECWNNNVNQRPSFRDLALRVDQIRDN

(19) United States
(12) Patent Application Publication
(10) Pub. No.: US 2025/0179431 A1
(43) Pub. Date: Jun. 5, 2025

(54) LIVE CELL CONSTRUCTS FOR PRODUCTION OF CULTURED MILK PRODUCT AND METHODS USING THE SAME

(71) Applicant: BIOMILQ, Inc., Durham, NC (US)

(72) Inventor: Leila Strickland, Hillsborough, NC (US)

(21) Appl. No.: 19/048,524

(22) Filed: Feb. 7, 2025

Related U.S. Application Data
(63) Continuation of application No. 17/467,358, filed on Sep. 6, 2021, now abandoned.
Application No. 17/467,358 is a continuation of application No. 17/247,672, filed on Dec. 18, 2020, now Pat. No. 11,111,477.
(60) Provisional application No. 63/199,164, filed on Dec. 10, 2020.
(60) Provisional application No. 62/958,407, filed on Jan. 8, 2020.

(51) Int. Cl. C12N 5/071; A23C 9/00; A61K 35/20

(52) U.S. Cl. CPC C12N 5/0631; A23C 9/00; A61K 35/20; C12N 2500/02; C12N 2513/00; C12N 2523/00; C12N 2533/90

(57) ABSTRACT

This invention relates to live cell constructs for in vitro and/or ex vivo production of cultured milk products from mammary cells, methods of producing isolated cultured milk products from mammary cells, bioreactors for producing isolated cultured milk products, and cultured milk products.

### CROSS-REFERENCE

This application is a continuation of U.S. application Ser. No. 17/467,358, filed Sep. 6, 2021, which is a continuation of U.S. application Ser. No. 17/247,672, filed on Dec. 18, 2020, now issued as U.S. Pat. No. 11,111,477 on Sep. 7, 2021, which claims the benefit of priority from U.S. Provisional Application No. 62/958,407, filed on Jan. 8, 2020, and U.S. Provisional Application No. 63/199,164, filed on Dec. 10, 2020, the contents of each are incorporated herein by reference in their entireties.

### SEQUENCE LISTING

The contents of the electronic sequence listing (BMQ-001USC2_SL.xml; Size: 8,242 bytes; and Date of Creation: Feb. 6, 2025) is herein incorporated by reference in its entirety.

### FIELD OF THE INVENTION

This invention relates to live cell constructs and methods using the same for in vitro and/or ex vivo production of cultured milk product from cultured mammary cells.

### BACKGROUND OF THE INVENTION

Milk is a staple of the human diet, both during infancy and throughout life. The American Academy of Pediatrics and World Health Organization recommend that infants be exclusively breastfed for the first 6 months of life, and consumption of dairy beyond infancy is a mainstay of human nutrition, representing a 700 billion dollar industry worldwide. However, lactation is a physiologically demanding and metabolically intensive process that can present biological and practical challenges for breastfeeding mothers, and milk production is associated with environmental, social, and animal welfare impacts in agricultural contexts.

The possibility of using mammalian cell culture to produce food has gained increasing interest in recent years, with the development of several successful prototypes of meat and sea food products from cultured muscle and fat cells (Stephens et al. 2018 Trends Food Sci Technol. 78:155-166). Additionally, efforts are underway to commercialize the production of egg and milk proteins using microbial expression systems. However, this fermentation-based process relies on the genetically engineered expression and purification of individual components and is unable to reproduce the full molecular profile of milk or dairy.

The present invention overcomes shortcomings in the art by providing live cell constructs and methods using the same for in vitro and/or ex vivo production of cultured milk product from cultured mammary cells.

### SUMMARY OF THE INVENTION

Disclosed herein, in certain embodiments, are live cell constructs, comprising: (a) a three-dimensional scaffold having an exterior surface, an interior surface defining an interior cavity/basal chamber, and a plurality of pores extending from the interior surface to the exterior surface; (b) a matrix material disposed on the exterior surface of the three-dimensional scaffold; (c) a culture media disposed within the interior cavity/basal chamber and in fluidic contact with the internal surface; and (d) an at least 70% confluent monolayer of polarized mammary cells disposed on the matrix material, wherein the mammary cells are selected from the group consisting of: live primary mammary epithelial cells, live mammary myoepithelial cells, live mammary progenitor cells, live immortalized mammary epithelial cells, live immortalized mammary myoepithelial cells, and live immortalized mammary progenitor cells. In some embodiments, the polarized mammary cells comprise an apical surface and a basal surface. In some embodiments, the basal surface of the mammary cells is in fluidic contact with the culture media. In some embodiments, at least 70%, at least 80%, at least 90%, at least 95%, at least 99%, or 100% of the mammary cells are polarized in the same orientation. In some embodiments, the monolayer of polarized mammary cells is at least 70% confluent, at least 80% confluent, at least 90% confluent, at least 95% confluent, at least 99% confluent, or 100% confluent. In some embodiments, the mammary cells comprise a constitutively active prolactin receptor protein. In some embodiments, the culture medium comprises a carbon source, a chemical buffering system, one or more essential amino acids, one or more vitamins and/or cofactors, and one or more inorganic salts. In some embodiments, the culture medium further comprises prolactin. In some embodiments, the matrix material comprises one or more extracellular matrix proteins. In some embodiments, the three-dimensional scaffold comprises a natural polymer, a biocompatible synthetic polymer, a synthetic peptide, a composite derived from any of the preceding, or any combination thereof. In some embodiments, the natural polymer is collagen, chitosan, cellulose, agarose, alginate, gelatin, elastin, heparan sulfate, chondroitin sulfate, keratan sulfate, and/or hyaluronic acid. In some embodiments, the biocompatible synthetic polymer is polysulfone, polyvinylidene fluoride, polyethylene co-vinyl acetate, polyvinyl alcohol, sodium polyacrylate, an acrylate polymer, and/or polyethylene glycol.

Disclosed herein, in certain embodiments, are methods of producing an isolated cultured milk product from mammary cells, the method comprising: (a) culturing a live cell construct in a bioreactor under conditions which produce the cultured milk product, said live cell construct comprising: (i) a three-dimensional scaffold having an exterior surface, an interior surface defining an interior cavity/basal chamber, and a plurality of pores extending from the interior surface to the exterior surface; (ii) a matrix material disposed on the exterior surface of the three-dimensional scaffold; (iii) a culture media disposed within the interior cavity/basal chamber and in fluidic contact with the internal surface; and (iv) an at least 70% confluent monolayer of polarized mammary cells disposed on the matrix material, wherein the mammary cells are selected from the group consisting of: live primary mammary epithelial cells, live mammary myoepithelial cells, live mammary progenitor cells, live immortalized mammary epithelial cells, live immortalized mammary myoepithelial cells, and live immortalized mammary progenitor cells; and (b) isolating the cultured milk product. In some embodiments, the polarized mammary cells comprise an apical surface and a basal surface. In some embodiments, the basal surface of the mammary cells is in fluidic contact with the culture media. In some embodiments, the bioreactor is an enclosed bioreactor. In some embodiments, the bioreactor comprises an apical compartment that is substantially isolated from the internal cavity/basal chamber of the live cell construct. In some embodiments, the apical compartment is in fluidic contact with the apical surface of the mammary cells. In some embodiments, the cultured milk product is secreted from the apical surface of the mammary cells into the apical compartment. In some embodiments, the culture media substantially does not contact the cultured milk product. In some embodiments, the total cell density of mammary cells within the bioreactor is at least 10¹¹. In some embodiments, the total surface area of mammary cells within the bioreactor is at least 1.5 m². In some embodiments, the culture medium comprises a carbon source, a chemical buffering system, one or more essential amino acids, one or more vitamins and/or cofactors, and one or more inorganic salts. In some embodiments, the matrix material comprises one or more extracellular matrix proteins. In some embodiments, the scaffold comprises a natural polymer, a biocompatible synthetic polymer, a synthetic peptide, a composite derived from any of the preceding, or any combination thereof. In some embodiments, the natural polymer is collagen, chitosan, cellulose, agarose, alginate, gelatin, elastin, heparan sulfate, chondroitin sulfate, keratan sulfate, and/or hyaluronic acid. In some embodiments, the biocompatible synthetic polymer is polysulfone, polyvinylidene fluoride, polyethylene co-vinyl acetate, polyvinyl alcohol, sodium polyacrylate, an acrylate polymer, and/or polyethylene glycol. In some embodiments, the culturing is carried out at a temperature of about 27° C. to about 39° C. In some embodiments, the culturing is carried out at a temperature of about 30° C. to about 37° C. In some embodiments, the culturing is carried out at an atmospheric concentration of CO₂ of about 4% to about 6%. In some embodiments, the culturing is carried out at an atmospheric concentration of CO₂ of about 5%.

Disclosed herein, in certain embodiments, are bioreactors, comprising: (a) an apical compartment comprising a cultured milk product; and (b) at least one live cell construct comprising: (i) a three-dimensional scaffold having an exterior surface, an interior surface defining an interior cavity/basal chamber, and a plurality of pores extending from the interior surface to the exterior surface; (ii) a matrix material disposed on the exterior surface of the three-dimensional scaffold; (iii) a culture media disposed within the interior cavity/basal chamber and in fluidic contact with the internal surface; and (iv) an at least 70% confluent monolayer of polarized mammary cells disposed on the matrix material, wherein the mammary cells are selected from the group consisting of: live primary mammary epithelial cells, live mammary myoepithelial cells, live mammary progenitor cells, live immortalized mammary epithelial cells, live immortalized mammary myoepithelial cells, and live immortalized mammary progenitor cells; wherein the apical surface of the mammary cells is in fluidic contact with the apical compartment. In some embodiments, the total cell density of mammary cells within the bioreactor is at least 10¹¹. In some embodiments, the total surface area of mammary cells within the bioreactor is at least 1.5 m².

Disclosed herein, in certain embodiments, are live cell constructs comprising mammary cells that compartmentalize feeding of the cells and secretion of cultured milk product.

Disclosed herein, in certain embodiments, are live cell constructs comprising, a scaffold having a top surface and a bottom surface; and a continuous monolayer of (a) live primary mammary epithelial cells, (b) a mixed population of live primary mammary epithelial cells, mammary myoepithelial cells and mammary progenitor cells, and/or (c) live immortalized mammary epithelial cells on the top surface of the scaffold, the continuous monolayer of (a) live primary mammary epithelial cells, (b) mixed population of live primary mammary epithelial cells, mammary myoepithelial cells and mammary progenitor cells, and/or (c) immortalized mammary epithelial cells having an apical surface and a basal surface (e.g., the cells form a polarized and confluent cell monolayer), wherein the construct comprises an apical compartment above and adjacent to the apical surface of the continuous monolayer of the (a) live primary mammary epithelial cells, the (b) mixed population of live primary mammary epithelial cells, mammary myoepithelial cells and mammary progenitor cells, and/or the (c) immortalized mammary epithelial cells and a basal compartment below and adjacent to the bottom surface of the scaffold.

Disclosed herein, in certain embodiments, are methods of producing milk in culture, the method comprising culturing the live cell construct of the present invention, thereby producing milk in culture.

Disclosed herein, in certain embodiments, are methods of making a live cell construct for producing milk in culture, the method comprising (a) isolating primary mammary epithelial cells, myoepithelial cells and/or mammary progenitor cells from mammary explants from mammary tissue (e.g., breast, udder, teat tissue), biopsy sample, or raw breastmilk, to produce isolated mammary epithelial cells, myoepithelial cells and mammary progenitor cells; (b) culturing the isolated primary mammary epithelial cells, myoepithelial cells and mammary progenitor cells to produce a mixed population of primary mammary epithelial cells, mammary myoepithelial cells and mammary progenitor cells; (c) cultivating the mixed population of (b) on a scaffold, the scaffold having an upper surface and lower surface, to produce a polarized, continuous (i.e., confluent) monolayer of primary mammary epithelial cells, myoepithelial cells and mammary progenitor cells of the mixed population on the upper surface of the scaffold, wherein the polarized, continuous monolayer comprises an apical surface and a basal surface, thereby producing a live cell construct for producing milk in culture.

Disclosed herein, in certain embodiments, are methods of making a live cell construct for producing milk in culture, the method comprising: a) isolating primary mammary epithelial cells, myoepithelial cells, and/or mammary progenitor cells from mammary explants from mammary tissue (e.g., breast, udder, teat tissue), biopsy sample, or raw breastmilk, to produce isolated mammary epithelial cells, myoepithelial cells, and/or mammary progenitor cells; (b) culturing the isolated primary mammary epithelial cells, myoepithelial cells, and/or mammary progenitor cells to produce a mixed population of primary mammary epithelial cells, mammary myoepithelial cells and mammary progenitor cells; (c) sorting the mixed population of primary mammary epithelial cells, myoepithelial cells, and/or mammary progenitor cells to produce a population of primary mammary epithelial cells; and (d) cultivating the population of primary mammary epithelial cells on a scaffold, the scaffold having an upper surface and lower surface, to produce a polarized, continuous (i.e., confluent) monolayer of primary mammary epithelial cells on the upper surface of the scaffold, wherein the polarized, continuous monolayer comprises an apical surface and a basal surface, thereby producing a live cell construct for producing milk in culture.

Disclosed herein, in certain embodiments, are methods of making a live cell construct for producing milk in culture, the method comprising (a) culturing immortalized mammary epithelial cells to produce increased numbers of immortalized mammary epithelial cells; (b) cultivating the immortalized mammary epithelial cells of (a) on a scaffold, the scaffold having an upper surface and lower surface, to produce a polarized, continuous (i.e., confluent) monolayer of immortalized mammary epithelial cells on the upper surface of the scaffold, wherein the polarized, continuous monolayer comprises an apical surface and a basal surface, thereby producing a live cell construct for producing milk in culture.

Disclosed herein, in certain embodiments, are methods of producing milk in culture comprising, culturing a live cell construct comprising (a) a scaffold comprising an upper surface and a lower surface and a continuous (i.e., confluent) polarized monolayer of live primary mammary epithelial cells, a continuous polarized monolayer of a mixed population of live primary mammary epithelial cells, mammary myoepithelial cells and mammary progenitor cells, and/or a continuous polarized monolayer of live immortalized mammary epithelial cells having an apical surface and a basal surface, wherein the continuous polarized monolayer of live primary mammary epithelial cells, the continuous polarized monolayer of the mixed population of live primary mammary epithelial cells, mammary myoepithelial cells and mammary progenitor cells and/or the continuous polarized monolayer of live immortalized mammary epithelial cells are located on the upper surface of the scaffold, (b) a basal compartment and an apical compartment, wherein the lower surface of the scaffold is adjacent to the basal compartment and the apical surface of the monolayer of live primary mammary epithelial cells, the monolayer of the mixed population of live primary mammary epithelial cells, mammary myoepithelial cells and mammary progenitor cells, and/or the monolayer of live immortalized mammary epithelial cells is adjacent to the apical compartment, wherein the monolayer of live primary epithelial mammary cells, the live primary epithelial mammary cells of the monolayer of the mixed population of live primary mammary epithelial cells, mammary myoepithelial cells and mammary progenitor cells, or the monolayer of immortalized mammary epithelial cells secretes milk through its apical surface into the apical compartment, thereby producing milk in culture.

Disclosed herein, in certain embodiments, are methods of producing a modified primary mammary epithelial cell or an immortalized mammary epithelial cell, wherein the method comprises introducing into the cell: (a) a polynucleotide encoding a prolactin receptor comprising a modified intracellular signaling domain, optionally wherein the prolactin receptor comprises a truncation wherein position 154 of exon 10 has been spliced to the 3′ sequence of exon 11; (b) a polynucleotide encoding a chimeric prolactin receptor that binds to a ligand, which is capable of activating milk synthesis in the absence of prolactin; (c) a polynucleotide encoding a constitutively or conditionally active prolactin receptor protein, optionally wherein the polynucleotide encodes a constitutively active human prolactin receptor protein comprising a deletion of amino acids 9 through 187; (d) a polynucleotide encoding a modified (recombinant) effector of a prolactin protein comprising (i) a JAK2 tyrosine kinase domain fused to a STAT5 tyrosine kinase domain; and/or (ii) a prolactin receptor intracellular domain fused to a JAK2 tyrosine kinase domain; (c) a loss of function mutation into a circadian related gene PER2 (period circadian protein homolog 2); and/or (f) a polynucleotide encoding one or more glucose transporter genes GLUT1 and/or GLUT12, thereby increasing the rate of nutrient uptake at the basal surface of a monolayer of cells of the modified primary mammary epithelial cell or immortalized mammary epithelial cell.

### BRIEF DESCRIPTION OF THE DRAWINGS

FIG. 1 shows an example of the collection of milk for nutritional use from mammary epithelial cells grown as a confluent monolayer in a compartmentalizing culture apparatus in which either fresh or recycled media is provided to the basal compartment and milk is collected from the apical compartment. TEER, transepithelial electrical resistance.

FIG. 2 shows an example of polarized absorption of nutrients and secretion of milk across a confluent monolayer of mammary epithelial cells anchored to a scaffold at the basal surface.

FIG. 3 shows an example micropatterned scaffold that provides increased surface area for the compartmentalized absorption of nutrients and secretion of milk by a confluent monolayer of mammary epithelial cells.

FIG. 4 shows three examples of a hollow fiber bioreactor depicted as a bundle of capillary tubes (top), which can support mammary epithelial cells lining either the external (top and lower left) or internal (lower right) surface of the capillaries, providing directional and compartmentalized absorption of nutrients and secretion of milk.

FIG. 5 exemplifies a cross-section of three-dimensional live cell construct. The construct is made up of a scaffold having an interior surface defining an interior cavity/basal chamber and an exterior surface. The interior cavity/basal chamber comprises cell culture media. A matrix material sits on top of the exterior surface of the scaffold. Pores transverse the scaffold from the interior surface to the exterior surface, allowing cell media to contact the basal surface of the cells of the cell monolayer disposed on the matrix material.

FIG. 6 exemplifies a bioreactor for producing a cultured milk product. The bioreactor is made up of a live cell construct and an apical chamber. The cell construct is made up of a scaffold having an interior surface defining an interior cavity/basal chamber and an exterior surface. The cavity comprises cell culture media. A matrix material sits on top of the exterior surface of the scaffold. Pores transverse the scaffold from the interior surface to the exterior surface, allowing cell media to contact the basal surface of the cells of the cell monolayer disposed on the matrix material. The apical surface of the cells of the cell monolayer secrete the milk/cultured milk product into the apical chamber. The apical chamber and the interior cavity/basal chamber are separated by the cell monolayer.

FIG. 7 exemplifies a live cell construct. The construct is made up of a scaffold having an interior surface defining an interior cavity/basal chamber and an exterior surface. The interior cavity/basal chamber comprises cell culture media. A matrix material sits on top of the exterior surface of the scaffold. Pores transverse the scaffold from the interior surface to the exterior surface, allowing cell media to contact the basal surface of the cells of the cell monolayer disposed on the matrix material.

### DETAILED DESCRIPTION OF THE INVENTION

Milk is a nutrient-rich liquid food produced in the mammary glands of mammals. It is a primary source of nutrition for infant mammals (including humans who are breastfed) before they are able to digest other types of food. Human milk is not merely nutrition. Rather, human milk contains a variety of factors with bioactive qualities that have a profound role in infant survival and health. Natural milk contains many other macronutrients, including proteins, lipids, polysaccharides and lactose. Milk consumption occurs in two distinct overall types: a natural source of nutrition for all infant mammals and a food product.

In almost all mammals, milk is fed to infants through breastfeeding, either directly or by expressing the milk to be stored and consumed later. Early milk from mammals contains antibodies that provide protection to the newborn baby as well as nutrients and growth factors. Breast milk is not a uniform, unvarying, constant, factory-made product; rather, it is a biological product produced by women with markedly varying genotypes, phenotypes, and diets. To add to the complexity, the composition of breast milk is influenced by a myriad of maternal, infant, and environmental factors. Human milk contains a rich array of proteins, carbohydrates, lipids, fatty acids, minerals, and vitamins, but most of its disease-fighting potential comes from a plethora of antibodies, leukocytes, hormones, antimicrobial peptides, cytokines, chemokines, and other bioactive factors.

Mammary epithelial cells (MECs) in culture have been previously demonstrated to display organization and behavior similar to that observed in vivo (Arevalo et al. 2016 Am J Physiol Cell Physiol. 310(5):C348-3 56; Chen et al. 2019 Curr Protoc Cell Biol. 82(1):e65). In Arevalo et al., specific biomarkers of MEC populations were detected in immortalized bovine mammary epithelial cells (BME-UV1) and immortalized bovine mammary alveolar cells (MAC-T) cultured on adherent 2-D plates, ultralow attachment surface 3D microplates, and 3D plates coated with Matrigel. Additionally, in Chen et al., protocols are detailed for isolation and culture of human primary mammary epithelial stem/progenitor cells from human breast tissue and subsequent generation of mammospheres using 3D organoid culture on gelatin sponges and Matrigel matrices. However, neither Arevalo nor Chen attempted to stimulate the production of milk from these MEC cultures.

In particular, when grown on an appropriate extracellular matrix and stimulated with prolactin, cultured bovine mammary epithelial cells polarize and organize into structures capable of secreting certain milk components (Blatchford et al. 1999 Animal Cell Technology: Basic & Applied Aspects 10:141-145). In Blatchford et al, bovine MECs polarized and formed mammospheres. Casein and butyrophilin were isolated from the cultures. However, the cells did not polarize in one uniform direction. Blatchford, et al. noted that the milk proteins were distributed in between the cells and dispersed throughout the mammospheres. Due to the lack of a uniform polarization orientation, Blatchford had to isolate the secreted proteins from the culture media.

Furthermore, in vitro two-dimensional models, such as those used in Blatchford et al. provide a low surface area-to-volume ratio (low density format). The surface area available for cell attachment limits the number of cells that can be grown

The only known attempt to culture mouse mammary epithelial cells in a high-density format, such as the hollow fiber bioreactor, failed to achieve compartmentalization necessary for the production and extraction of a cultured milk product (Sharfstein et al. 1992 Biotechnology and Bioengineering 40:672-680). In Sharfstein et al., growth, long-term expression of functional differentiation, and metabolism of COMMA-ID (an immortalized mouse mammary epithelial cell line) was examined in two different systems: extended batch culture and hollow-fiber reactor culture. Using COMMA-ID seeded onto Costar Transwell® polycarbonate membrane cell culture inserts, Sharfstein et al. created a confluent monolayer capable of barrier formation and polarized metabolism between the apical and basal side that maintained gradients of glucose and lactate. However, using a hollow-fiber bioreactor culture, Sharfstein et al. was unable to achieve separation of basal and apical compartments. Furthermore, it was not determined if nutrient uptake was polarized in a hollow-fiber culture (Sharfstein et al. 1992). Importantly, no prior work has been able to culture mammary epithelial cells from humans or other nutritionally relevant species in a high-density, three-dimensional, compartmentalizing format.

Disclosed herein, in certain embodiments, are live cell constructs, methods of making the same, and methods of using the same for in vitro and/or ex vivo production of cultured milk product from cultured mammary cells.

This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

Unless the context indicates otherwise, it is specifically intended that the various features described herein can be used in any combination. Moreover in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

### Definitions

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

Moreover, any feature or combination of features set forth herein can be excluded or omitted.

The term “about,” as used herein when referring to a measurable value such as an amount of a compound or agent, dose, time, temperature, and the like, is meant to encompass variations of ±10%, ±5%, ±1%, ±0.5%, or even ±0.1% of the specified amount.

Unless otherwise defined, all technical and scientific terms used herein have the same meaning as commonly understood by one of ordinary skill in the art. The terminology used in the description herein is for the purpose of describing particular embodiments only and is not intended to be limiting.

Nucleotide sequences are presented herein by single strand only, in the 5′ to 3′ direction, from left to right, unless specifically indicated otherwise. Nucleotides and amino acids are represented herein in the manner recommended by the IUPAC-1UB Biochemical Nomenclature Commission, or (for amino acids) by either the one-letter code, or the three letter code, both in accordance with 37 C.F.R. § 1.822 and established usage.

Except as otherwise indicated, standard methods known to those skilled in the art may be used for production of recombinant and synthetic polypeptides, antibodies or antigen-binding fragments thereof, manipulation of nucleic acid sequences, production of transformed cells, the construction of viral vector constructs, and transiently and stably transfected packaging cells. Such techniques are known to those skilled in the art. See, e.g., Sambrook et al., Molecular Cloning: A Laboratory Manual 2nd Ed. (Cold Spring Harbor, NY, 1989); F. M. Ausubel et al. Current Protocols In Molecular Biology (Green Publishing Associates, Inc. and John Wiley & Sons, Inc., New York).

As used herein, the transitional phrase “consisting essentially of” is to be interpreted as encompassing the recited materials or steps and those that do not materially affect the basic and novel characteristic(s) of the claimed invention. Thus, the term “consisting essentially of” as used herein should not be interpreted as equivalent to “comprising.”

As used herein, the term “polypeptide” encompasses both peptides and proteins, and does not require any particular amino acid length or tertiary structure unless indicated otherwise.

The term “polarized” as used herein in reference to cells and/or monolayers of cells refers to a spatial status of the cell wherein there are two distinct surfaces of the cell, e.g., an apical surface and a basal surface, which may be different. In some embodiments, the distinct surfaces of a polarized cell comprises different surface and/or transmembrane receptors and/or other structures. In some embodiments, individual polarized cells in a continuous monolayer have similarly-oriented apical surfaces and basal surfaces. In some embodiments, individual polarized cells in a continuous monolayer have communicative structures between individual cells (e.g., tight junctions) to allow cross communication between individual cells and to create separation (e.g., compartmentalization) of the apical compartment and basal compartment.

As used herein, “apical surface” means the surface of a cell that faces an external environment or toward a cavity or chamber, for example the cavity of an internal organ. With respect to mammary epithelial cells, the apical surface is the surface from which the cultured milk product is secreted.

As used herein, “basal surface” means the surface of a cell that is in contact with a surface, e.g., the matrix of a bioreactor.

As used herein, “bioreactor” means a device or system that supports a biologically active environment that enables the production of a culture milk product described herein from mammary cells described herein.

The term “lactogenic” as used herein refers to the ability to stimulate production and/or secretion of milk. A gene or protein (e.g., prolactin) may be lactogenic, as may any other natural and/or synthetic product. In some embodiments, a lactogenic culture medium comprises prolactin, thereby stimulating production of milk by cells in contact with the culture medium.

As used herein, the term “food grade” refers to materials considered non-toxic and safe for consumption (e.g., human and/or other animal consumption), e.g., as regulated by standards set by the U.S. Food and Drug Administration.

In some embodiments, milk produced by the primary mammary epithelial cells (e.g., primary mammary epithelial cells from the isolated live primary mammary epithelial cells and/or the primary mammary epithelial cells from the mixed population of live primary mammary epithelial cells, mammary myoepithelial cells and/or mammary progenitor cells) or the immortalized mammary epithelial cells is secreted through the apical surface of the cells into the apical compartment. In some embodiments, a basal compartment comprises a culture medium and the culture medium is in contact with the basal surface of the live primary mammary epithelial cells, the mixed population of live primary mammary epithelial cells, mammary myoepithelial cells and mammary progenitor cells, and/or the immortalized mammary epithelial cells.

### Live Cell Constructs

Disclosed herein, in certain embodiments, are live cell constructs for producing milk in culture, the live cell constructs comprising a continuous monolayer of live mammary cells selected from the group consisting of: (a) live primary mammary epithelial cells, (b) live mammary myoepithelial cells, (c) live mammary progenitor cells, and/or (d) live immortalized mammary epithelial cells.

In some embodiments, the mammary cells comprise milk-producing mammary epithelial cells, contractile myoepithelial cells, and/or progenitor cells that can give rise to both mammary epithelial and mammary contractile myoepithelial cells. Mammary epithelial cells are the only cells that produce milk. In some embodiments, the mammary cells comprise mammary epithelial cells, primary mammary epithelial cells, mammary myoepithelial cells and mammary progenitor cells.

In some embodiments, the mammary cells are from breast tissue, udder tissue, and/or teat tissue of a mammal. In some embodiments, the mammary cells are from any mammal, e.g., a primate (e.g., chimpanzee, orangutan, gorilla, monkey (e.g., Old World, New World), lemur, human), a dog, a cat, a rabbit, a mouse, a rat, a horse, a cow, a goat, a sheep, an ox (e.g., Bos spp.), a pig, a deer, a musk deer, a bovid, a whale, a dolphin, a hippopotamus, an elephant, a rhinoceros, a giraffe, a zebra, a lion, a cheetah, a tiger, a panda, a red panda, and an otter. In some embodiments, the mammary cells are from an endangered species, e.g., an endangered mammal. In some embodiments, the mammary cells are from a human. In some embodiments, the mammary cells are from a bovid (e.g., a cow).

In some embodiments, the continuous monolayer of live mammary cells is derived from breast milk-derived stem cells or breast stem cells originating from tissue biopsy of the mammary gland. The epithelial component of breast milk includes not only mature epithelial cells, but also their precursors and stem cells in culture. A subpopulation of breast milk-derived stem cells displays very high multilineage potential, resembling those typical for human embryonic stem cells (hESCs). Breast stem cells may also originate from tissue biopsy of the mammary gland, and include terminally differentiated MECs. Both breast milk-derived stem cells and breast stem cells originating from tissue biopsy of the mammary gland are multi-potent cells that can give rise to MECs or myoepithelial cells.

In some embodiments, at least 50% of the mammary cells of the live cells culture are polarized. In some embodiments, at least 55% of the mammary cells of the live cells culture are polarized. In some embodiments, at least 60% of the mammary cells of the live cells culture are polarized. In some embodiments, at least 65% of the mammary cells of the live cells culture are polarized. In some embodiments, at least 70% of the mammary cells of the live cells culture are polarized. In some embodiments, at least 75% of the mammary cells of the live cells culture are polarized. In some embodiments, at least 80% of the mammary cells of the live cells culture are polarized. In some embodiments, at least 85% of the mammary cells of the live cells culture are polarized. In some embodiments, at least 90% of the mammary cells of the live cells culture are polarized. In some embodiments, at least 95% of the mammary cells of the live cells culture are polarized. In some embodiments, at least 100% of the mammary cells of the live cells culture are polarized. In some embodiments, substantially all of the mammary cells of the live cell construct are polarized (i.e., have an apical surface and a basal surface). In some embodiments, substantially all of the mammary cells of the live cell construct are polarized and substantially all of the polarized cells are oriented in the same direction. For example, in some embodiments, substantially all of the mammary cells have an apical surface and a basal surface, wherein the apical surface of substantially all of the cells is oriented in the same direction and the basal surface of substantially all of the cells is oriented in the same direction.

In some embodiments, the monolayer of epithelial mammary cells has at least 70% confluence over the scaffold. In some embodiments, the monolayer of mammary epithelial cells has at least about 75% confluence over the scaffold. In some embodiments, the monolayer of epithelial mammary cells has at least about 80% confluence over the scaffold. In some embodiments, the monolayer of epithelial mammary cells has at least about 85% confluence over the scaffold. In some embodiments, the monolayer of epithelial mammary cells has at least about 90% confluence over the scaffold. In some embodiments, monolayer of epithelial mammary cells has at least about 95% confluence over the scaffold. In some embodiments, the monolayer of epithelial mammary cells has at least about 99% confluence over the scaffold. In some embodiments, the monolayer of epithelial mammary cells has 100% confluence over the scaffold.

### Genetic Modifications to Mammary Cells

In some embodiments, the mammary cells comprise a constitutively active prolactin receptor protein. In some embodiments, the mammary cells comprise a constitutively active human prolactin receptor protein. Where the primary mammary epithelial cell or immortalized mammary epithelial cells comprise a constitutively active prolactin receptor, the culture medium does not contain prolactin.

In some embodiments, the constitutively active human prolactin receptor protein comprises a deletion of amino acids 9 through 187, wherein the numbering is based on the reference amino acid sequence of a human prolactin receptor identified as SEQ ID NO: 1.

SEQ ID NO: 1: Human prolactin receptor (GenBank
accession number AAD32032.1)
MKENVASATVFTLLLFLNTCLLNGQLPPGKPEIFKCRSPNKETFTCWWR
PGTDGGLPTNYSLTYHREGETLMHECPDYITGGPNSCHFGKQYTSMWRT
YIMMVNATNQMGSSFSDELYVDVTYIVQPDPPLELAVEVKQPEDRKPYL
WIKWSPPTLIDLKTGWFTLLYEIRLKPEKAAEWEIHFAGQQTEFKILSL
HPGQKYLVQVRCKPDHGYWSAWSPATFIQIPSDFTMNDTTVWISVAVLS
AVICLIIVWAVALKGYSMVTCIFPPVPGPKIKGFDAHLLEKGKSEELLS
ALGCQDFPPTSDYEDLLVEYLEVDDSEDQHLMSVHSKEHPSQGMKPTYL
DPDTDSGRGSCDSPSLLSEKCEEPQANPSTFYDPEVIEKPENPETTHTW
DPQCISMEGKIPYFHAGGSKCSTWPLPQPSQHNPRSSYHNITDVCELAV
GPAGAPATLLNEAGKDALKSSQTIKSREEGKATQQREVESFHSETDQDT
PWLLPQEKTPFGSAKPLDYVEIHKVNKDGALSLLPKQRENSGKPKKPGT
PENNKEYAKVSGVMDNNILVLVPDPHAKNVACFEESAKEAPPSLEQNQA
EKALANFTATSSKCRLQLGGLDYLDPACFTHSFH

In some embodiments, the constitutively active human prolactin receptor protein comprising a deletion of the following amino acids:

VFTLLLFLNTCLLNGQLPPGKPEIFKCRSPNKETFTCWWRPGTDGGLPTN
YSLTYHREGETLMHECPDYITGGPNSCHFGKQYTSMWRTYIMMVNATNQM
GSSFSDELYVDVTYIVQPDPPLELAVEVKQPEDRKPYLWIKWSPPTLIDL
KTGWFTLLYEIRLKPEKAA (e.g., amino acid positions 10
through 178 of SEQ ID NO: 1).

In some embodiments, the mammary cells comprise a loss of function mutation introduced into a circadian related gene PER2. In some embodiments, the loss of function mutation introduced into a circadian related gene PER2 promotes increased synthesis of cultured milk components. In some embodiments, the loss of function mutation in the PER2 gene comprises an 87-amino acid deletion from position 348 to 434 in PER2, wherein the numbering is based on the reference amino acid sequence of a human PER2 identified as SEQ ID NO:2.

SEQ ID NO: 2: Human Period circadian protein
homolog 2 (GenBank accession number NM_022817)
MNGYAEFPPSPSNPTKEPVEPQPSQVPLQEDVDMSSGSSGHETNENCST
GRDSQGSDCDDSGKJELGMLVEPPDARQSPDTFSLMMAKSEHNPSTSGC
SSDQSSKVDTHKEL1KTLKELKVHLPADKKAKGKASTLATLKYALRSVK
QVKANEEYYQLLMSSEGHPCGADVPSYTVEEMESVTSEHIVKNADMFAV
AVSLVSGKILYISDQVASIFHCKRDAFSDAKFVEFLAPHDVGVFEISFT
SPYKLPLWSMCSGADSFTQECMEEKSFECRVSVRKSHENEIRYHPFRMT
PYLVKVRDQQGAESQLCCLLLAERVHSGYEAPRIPPEKRIFTTTHTPNC
LFQDVDERAVPLLGYLPQDLIETPVLVQLHPSDRPLMLAIHKKILQSGG
QPFDYSPIRFRARNGEYITLDTSWSSFINPWSRKISFIIGRHKVRVGPL
NEDVFAAHPCTEEKALHPSIQELTEQIHRLLLQPVPHSGSSGYGSLGSN
GSHEHLMSQTSSSDSNGHEDSRRRRAEICKNGNKTKNRSHYSHESGEQK
KKSVTEMQTNPPAEKKAVPAMEKDSLGVSFPEELACKNQPTCSYQQISC
LDSVIRYLESCNEAATLKRKCEFPANVPALRSSDKRKATVSPGPHAGEA
EPPSRVNSRTGVGTHLTSLALPGKAESVASLTSQCSYSSTIVHVGDKKP
QPELEMVEDAASGPESLDCLAGPALACGLSQEKEPFKKLGLTKEVLAAH
TQKEEQSFLQKFKEIRKLSIFQSHCHYYLQERSKGQPSERTAPGLRNTS
GIDSPWKKTGKNRKLKSKRVKPRDSSESTGSGGPVSARPPLVGLNATAW
SPSDTSQSSCPAVPFPAPVPAAYSLPVFPAPGTVAAPPAPPHASFTVPA
VPVDLQHQFAVQPPPFPAPLAPVMAFMLPSYSFPSGTPNLPQAFFPSQP
QFPSHPTLTSEMASASQPEFPEGGTGAMGTTGATETAAVGADCKPGTSR
DQQPKAPLTRDEPSDTQNSDALSTSSGLLNLLLNEDLCSASGSAASESL
GSGSLGCDASPSGAGSSDTSHTSKYEGSIDSSENNHKAKMNTGMEESEH
FIKCVLQDPIWLLMADADSSVMMTYQLPSRNLEAVLKEDREKLKLLQKL
QPRFTESQKQELREVHQWMQTGGLPAAIDVAECVYCENKEKGNICIPYE
EDIPSLGLSEVSDTKEDENGSPLNHRIEEQT

In some embodiments, the loss of function mutation introduced into PER2 comprises a deletion of the following amino acids:

CLFQDVDERAVPLLGYLPQDLIETPVLVQLHPSDRPLMLAIHKKILQSGG
QPFDYSPIRFRARNGEYITLDTSWSSFINPWSRKISFIIGRHKV
(e.g., amino acid positions 341 through 434 of SEQ
ID NO: 2).

In some embodiments, the mammary cells comprise a polynucleotide encoding a prolactin receptor comprising a modified intracellular signaling domain. In some embodiments, the loss of function mutation introduced into a circadian related gene PER2 promotes increased synthesis of individual cultured milk components. In some embodiments, the prolactin receptor comprises a truncation wherein position 154 of exon 10 has been spliced to the 3′ sequence of exon 11. In some embodiments, the prolactin receptor comprises a sequence according to SEQ ID NO: 3.

SEQ ID NO: 3: Human isoform 4 of Prolactin
receptor (GenBank accession number AF416619;
Trott et al. 2003 J. Mol. Endocrinol 3Q(I):
31-47)
MKENVASATVFTLLLFLNTCLLNGQLPPGKPEIFKCRSPNKETFTCWWR
PGTDGGLPTNYSLTYHREGETLMHECPDYITGGPNSCHFGKQYTSMWRT
YIMMVNATNQMGSSFSDELYVDVTYIVQPDPPLELAVEVKQPEDRKPYL
WIKWSPPTLIDLKTGWFTLLYEIRLKPEKAAEWEIHFAGQQTEFKILSL
HPGQKYLVQVRCKPDHGYWSAWSPATFIQIPSDFTMNDTTVWISVAVLS
AVICLIIVWAVALKGYSMVTCIFPPVPGPKIKGFDAHLLEKGKSEELLS
ALGCQDFPPTSDYEDLLVEYLEVDDSEDQHLMSVHSKEHPSQGDPLMLG
ASHYKNLKSYRPRKISSQGRLAVFTKATLTTVQ

In some embodiments, the mammary cells comprise a polynucleotide encoding a modified (e.g., recombinant) effector of a prolactin protein. In some embodiments, the modified effector of the prolactin protein comprises a janus kinase-2 (JAK2) tyrosine kinase domain. In some embodiments, the modified effector comprises a JAK2 tyrosine kinase domain fused to a signal transducer and activator of transcription-5 (STAT5) tyrosine kinase domain (e.g., a polynucleotide encoding a JAK2 tyrosine kinase domain linked to the 3′ end of a polynucleotide encoding the STAT5 tyrosine kinase domain). In some embodiments, the modified effector of a prolactin protein promotes increased synthesis of individual cultured milk components. In some embodiments, the modified effector has a sequence according to SEQ ID NO: 4. Bolded amino acids correspond to the JAK2 kinase domain of amino acid positions 757 through 1129 of a reference human JAK2 amino acid sequence.

STA5A Human signal transducer and activator of
transcription 5A fused at 3' end to amino acids
757-1129 of JAK2 human tyrosine-protein kinase.
SEQ ID NO: 4
MAGWIQAQQL QGDALRQMQV LYGQHFPIEV RHYLAQWIES
QPWDAIDLDN PQDRAQATQL LEGLVQELQK KAEHQVGEDG
FLLKIKLGHY ATQLQKTYDR CPLELVRCIR HILYNEQRLV
REANNCSSPA GILVDAMSQK HLQINQTFEE LRLVTQDTEN
ELKKLQQTQE YFIIQYQESL RIQAQFAQLA QLSPQERLSR
ETALQQKQVS LEAWLQREAQ TLQQYRVELA EKHQKTLQLL
RKQQTIILDD ELIQWKRRQQ LAGNGGPPEG SLDVLQSWCE
KLAEIIWQNR QQIRRAEHLC QQLPIPGPVE EMLAEVNATI
TDIISALVTS TFIIEKQPPQ VLKTQTKFAA TVRLLVGGKL
NVHMNPPQVK ATIISEQQAK SLLKNENTRN ECSGEILNNC
CVMEYHQATG TLSAHFRNMS LKRIKRADRR GAESVTEEKF
TVLFESQFSV GSNELVFQVK TLSLPWVIV HGSQDHNATA
TVLWDNAFAE PGRVPFAVPD KVLWPQLCEA LNMKFKAEVQ
SNRGLTKENL VFLAQKLFNN SSSHLEDYSG LSVSWSQFNR
ENLPGWNYTF WQWFDGVMEV LKKHHKPEIWN DGAILGFVNK
QQAHDLLINK PDGTFLLRFS DSEIGGITIA WKFDSPERNL
WNLKPFTTRD FSIRSLADRL GDLSYLIYVF PDRPKDEVFS
KYYTPVLAKA VDGYVKPQIK QWPEFVNAS ADAGGSSATY
MDQAPSPAVC PQAPYNMYPQ NPDHVLDQDG EFDLDETMDV
ARHVEELLRR PMDSLDSRLS PPAGLFTSAR GSLSLDSQ
RKLQFYEDRH QLPAPKWAEL ANLINNCMDY EPDFRPSFRA
IIRDLNSLFT PDYELLTEND MLPNMRIGAL GFSGAFEDRD
PTQFEERHLK FLQQLGKGNF GSVEMCRYDP LQDNTGEWA
VKKLQHSTEE HLRDFEREIE ILKSLQHDNI VKYKGVCYSA
GRRNLKLIME YLPYGSLRDY LQKHKERIDH IKLLQYTSQI
CKGMEYLGTK RYIHRDLATR NILVENENRV KIGDFGLTKV
LPQDKEYYKV KEPGESPIFW YAPESLTESK FSVASDVWSF
GWLYELFTY IEKSKSPPAE FMRMIGNDKQ GQMIVFHLIE
LLKNNGRLPR PDGCPDEIYM IMTECWNNNV NQRPSFRDLA
LRVDQIRDN.

In some embodiments, the mammary cells are immortalized. In some embodiments, the mammary cells comprise one or more nucleic acids encoding human telomerase reverse transcriptase (hTERT) or simian virus 40 (SV40). In some embodiments, the mammary cells comprise a small hairpin RNA (shRNA) to p16 (Inhibitor of Cyclin-Dependent Kinase 4) (p16(INK4)) and Master Regulator of Cell Cycle Entry and Proliferative Metabolism (c-MYC).

In some embodiments, the method comprises introducing into the cell: (a) a polynucleotide encoding a prolactin receptor comprising a modified intracellular signaling domain, optionally wherein the prolactin receptor comprises a truncation wherein position 154 of exon 10 has been spliced to the 3′ sequence of exon 11; (b) a polynucleotide encoding a chimeric prolactin receptor that binds to a ligand, which is capable of activating milk synthesis in the absence of prolactin; (c) a polynucleotide encoding a constitutively or conditionally active prolactin receptor protein, optionally wherein the polynucleotide encodes a constitutively active human prolactin receptor protein comprising a deletion of amino acids 9 through 187 (e.g., a deletion of amino acids 9 through 187, wherein the numbering is based on the reference amino acid sequence of a human prolactin receptor identified as SEQ ID NO: 1); (d) a polynucleotide encoding a modified (e.g., recombinant) effector of a prolactin protein comprising (i) a janus kinase-2 (JAK2) tyrosine kinase domain, optionally wherein the JAK2 tyrosine kinase domain is fused to a signal transducer and activator of transcription-5 (STAT5) tyrosine kinase domain (e.g., a polynucleotide encoding a JAK2 tyrosine kinase domain linked to the 3′ end of a polynucleotide encoding the STAT5 tyrosine kinase domain); and/or (ii) a prolactin receptor intracellular domain fused to a JAK2 tyrosine kinase domain; (e) a loss of function mutation into a circadian related gene PER2 (period circadian protein homolog 2); and/or (f) a polynucleotide encoding one or more glucose transporter genes GLUT1 and/or GLUT12, thereby increasing the rate of nutrient uptake at the basal surface of the monolayer.

### Scaffolds

In some embodiments, the live cell construct further comprises a scaffold having a top surface/exterior surface and a bottom surface/interior surface. In some embodiments, the scaffold is a 2-dimensional surface or a 3-dimensional surface (e.g., a 3-dimensional micropatterned surface, and/or as a cylindrical structure that is assembled into bundles). A non-limiting example of a 2-dimensional surface scaffold is a Transwell® filter. In some embodiments, the scaffold is a 3-dimensional surface. Non-limiting examples of a 3-dimensional micropatterned surface include a microstructured bioreactor, a decellularized tissue (e.g., a decellularized mammary gland or decellularized plant tissue), micropatterned scaffolds fabricated through casting or three-dimensional printing with biological or biocompatible materials, textured surface. In some embodiments, the scaffold is produced by electrospinning cellulose nanofibers and/or a cylindrical structure that can be assembled into bundles (e.g., a hollow fiber bioreactor). In some embodiments, the scaffold is porous. In some embodiments, the scaffold is a 3D scaffold. In some embodiments, the 3-dimensional scaffold is any structure which has an enclosed hollow interior/central cavity. In some embodiments, the three dimensional scaffold joins with one or more surfaces to form an enclosed interior chamber/basal compartment. For example, the scaffold can join with one or more walls of a bioreactor to form the interior chamber/basal compartment. In some embodiments, the scaffold is a hollow fiber bioreactor. In some embodiments, the 3D scaffold is a tube in which the central cavity is defined by the interior surface of the scaffold. In some embodiments, the 3D scaffold is a hollow sphere in which the central cavity is defined by the interior surface of the scaffold.

For in vitro culture methods for studies of intestinal absorption, 2-dimensional surface scaffold such as Transwells® have long been used as the standard as they provide both apical and basolateral spaces to simulate the gut-blood-barrier and enable both active and passive transport of drugs and nutrients. However, cells seeded onto flat supports exhibit markedly different phenotypes to cells in vivo, partly due to the poor representation of the 3-D extracellular microenvironments.

A 3-dimensional scaffold allows mammary cells (e.g., MECs) to grow or interact with their surroundings in all three dimensions. Unlike 2D environments, a 3D cell culture allows cells in vitro to grow in all directions, approximating the in vivo mammary environment. Further, the 3D scaffold allows for a larger surface area for culture of the cells and for metabolite and gas exchange, plus it enables necessary compartmentalization—enabling the cultured milk product to be secreted into one compartment, while the cell culture media is contacted with the mammary cells in another compartment. To date, a confluent monolayer with polarized separation of basal and apical cell surfaces using mammary epithelial cell on a 3D surface has not been achieved (Sharfstein et al. 1992).

In some embodiments, the scaffold is porous. In some embodiments, the scaffold is permeable to the cell media, allowing the cell media to contact the cells of the cell monolayer. In some embodiments, the scaffold is transversed by at least one pore that allows the cell media to contact the basal surface of the cells of the cell monolayer.

In some embodiments, the top surface/exterior surface of the scaffold is coated with a matrix material. In some embodiments, the matrix is made up of one or more extracellular matrix proteins. Non-limiting examples of extracellular matrix proteins include collagen, laminin, entactin, tenascin, and/or fibronectin. In some embodiments, the scaffold comprises a natural polymer, a biocompatible synthetic polymer, a synthetic peptide, and/or a composite derived from any combination thereof. In some embodiments, a natural polymer useful with this invention includes, but is not limited to, collagen, chitosan, cellulose, agarose, alginate, gelatin, elastin, heparan sulfate, chondroitin sulfate, keratan sulfate, and/or hyaluronic acid. In some embodiments, a biocompatible synthetic polymer useful with this invention includes, but is not limited to, cellulose, polysulfone, polyvinylidene fluoride, polyethylene co-vinyl acetate, polyvinyl alcohol, sodium polyacrylate, an acrylate polymer, and/or polyethylene glycol. In some embodiments, the top of the scaffold is coated with laminin and collagen.

In some embodiments, the matrix material is porous. In some embodiments, the matrix material is permeable to the cell media, allowing the cell media to contact the cells of the cell monolayer. In some embodiments, the matrix material is transversed by at least one pore that allows the cell media to contact the basal surface of the cells of the cell monolayer.

In some embodiments, the pore size of the scaffold and/or matrix material is at least about 0.1 μm. In some embodiments, the pore size of the scaffold and/or matrix material is at least about 0.2 μm. In some embodiments, the pore size of the scaffold and/or matrix material is at least about 0.3 μm. In some embodiments, the pore size of the scaffold and/or matrix material is at least about 0.4 μm. In some embodiments, the pore size of the scaffold and/or matrix material is at least about 0.5 μm. In some embodiments, the pore size of the scaffold and/or matrix material is at least about 0.6 μm. In some embodiments, the pore size of the scaffold and/or matrix material is at least about 0.7 μm. In some embodiments, the pore size of the scaffold and/or matrix material is at least about 0.8 μm. In some embodiments, the pore size of the scaffold and/or matrix material is at least about 0.9 μm. In some embodiments, the pore size of the scaffold and/or matrix material is at least about 1.0 μm. In some embodiments, the pore size of the scaffold and/or matrix material is at least about 1.1 μm. In some embodiments, the pore size of the scaffold and/or matrix material is at least about 1.2 μm. In some embodiments, the pore size of the scaffold and/or matrix material is at least about 1.3 μm. In some embodiments, the pore size of the scaffold and/or matrix material is at least about 1.4 μm. In some embodiments, the pore size of the scaffold and/or matrix material is at least about 1.5 μm. In some embodiments, the pore size of the scaffold and/or matrix material is at least about 1.6 μm. In some embodiments, the pore size of the scaffold and/or matrix material is at least about 1.7 μm. In some embodiments, the pore size of the scaffold and/or matrix material is at least about 1.8 μm. In some embodiments, the pore size of the scaffold and/or matrix material is at least about 1.9 μm. In some embodiments, the pore size of the scaffold and/or matrix material is at least about 2.0 μm. In some embodiments, the pore size of the scaffold and/or matrix material is at least about 2.1 μm. In some embodiments, the pore size of the scaffold and/or matrix material is at least about 2.2 μm. In some embodiments, the pore size of the scaffold and/or matrix material is at least about 2.2 μm. In some embodiments, the pore size of the scaffold and/or matrix material is at least about 2.3 μm. In some embodiments, the pore size of the scaffold and/or matrix material is at least about 2.4 μm. In some embodiments, the pore size of the scaffold and/or matrix material is at least about 2.5 μm. In some embodiments, the pore size of the scaffold and/or matrix material is at least about 2.6 μm. In some embodiments, the pore size of the scaffold and/or matrix material is at least about 2.7 μm. In some embodiments, the pore size of the scaffold and/or matrix material is at least about 2.8 μm. In some embodiments, the pore size of the scaffold and/or matrix material is at least about 2.9 μm. In some embodiments, the pore size of the scaffold and/or matrix material is at least about 3.0 μm.

In some embodiments, the live cell construct comprises a scaffold having a top surface/exterior surface and a bottom surface/interior surface; and a continuous monolayer of (a) live primary mammary epithelial cells, (b) a mixed population of live primary mammary epithelial cells, mammary myoepithelial cells and mammary progenitor cells, and/or (c) live immortalized mammary epithelial cells on the top surface of the scaffold, the continuous monolayer of (a) live primary mammary epithelial cells, (b) mixed population of live primary mammary epithelial cells mammary myoepithelial cells and mammary progenitor cells, and/or (c) immortalized mammary epithelial cells having an apical surface and a basal surface (e.g., the cells form a polarized and confluent cell monolayer), wherein the construct comprises an apical compartment above and adjacent to the apical surface of the continuous monolayer of the (a) live primary mammary epithelial cells, the (b) mixed population of live primary mammary epithelial cells, mammary myoepithelial cell and mammary progenitor cells, and/or the (c) immortalized mammary epithelial cells and a basal compartment below and adjacent to the bottom surface of the scaffold.

### Bioreactor

Disclosed herein, in certain embodiments, are bioreactors, comprising: (a) an apical compartment comprising a cultured milk product; and (b) at least one live cell construct comprising: (i) a three-dimensional scaffold having an exterior surface, an interior surface defining an interior cavity/basal chamber, and a plurality of pores extending from the interior surface to the exterior surface; (ii) a matrix material disposed on the exterior surface of the three-dimensional scaffold; (iii) a culture media disposed within the interior cavity/basal chamber and in fluidic contact with the internal surface; and (iv) an at least 70% confluent monolayer of polarized mammary cells disposed on the matrix material, wherein the mammary cells are selected from the group consisting of: live primary mammary epithelial cells, live mammary myoepithelial cells, live mammary progenitor cells, live immortalized mammary epithelial cells, live immortalized mammary myoepithelial cells, and live immortalized mammary progenitor cells; wherein the apical surface of the mammary cells is in fluidic contact with the apical compartment.

In some embodiments, the bioreactor is an enclosed bioreactor. In some embodiments, the apical chamber is substantially isolated from the interior cavity/basal compartment.

A hollow fiber bioreactor is an exemplary bioreactor for use with the methods disclosed here. The hollow fiber bioreactor is a high-density, continuous perfusion culture system that closely approximates the environment in which cells grow in vivo. It consists of thousands of semi-permeable 3D scaffolds (i.e., hollow fibers) in a parallel array within a cartridge shell fitted with inlet and outlet ports. These fiber bundles are potted or sealed at each end so that any liquid entering the ends of the cartridge will necessarily flow through the interior of the fibers. Cells are generally seeded outside the fibers within the cartridge in the extra capillary space (ECS).

Three fundamental characteristics differentiate hollow fiber cell culture from other methods: (1) cells are bound to a porous matrix much as they are in vivo, not a plastic dish, microcarrier or other impermeable support, (2) the molecular weight cut off of the support matrix can be controlled, and (3) extremely high surface area to volume ratio (150 cm² or more per mL) which provides a large area for metabolite and gas exchange for efficient growth of host cells.

The bioreactor structure provides a fiber matrix that allows permeation of nutrients, gases and other basic media components, as well as cell waste products, but not cells, where the cells can be amplified. Hollow fiber bioreactor technology has been used to obtain high density cell amplification by utilizing hollow fibers to create a semi-permeable barrier between the cell growth chamber and the medium flow. Since the surface area provided by this design is large, using this fiber as a culture substrate allows the production of large numbers of cells. Cells growing in the 3-dimensional environment within the bioreactor are bathed in fresh medium as it perfuses through the hollow fibers.

To replicate the topography of the intestine, Costello et al. developed a 3-D printed bioreactor that can both contain porous villus scaffolds via micromolding (Costello et al. 2017Scientific Reports 7(12515): 1-10). This geometrically complex molded scaffold provided separation of the apical and basolateral spaces in a manner in which fluid flow exposes intestinal epithelial cells to physiologically relevant shear stresses (Costello et al. 2017). Similarly, a long-term culture in vitro culture in a simulated gut-like environment was created by Morada et al. using a hollow fiber bioreactor which allowed for two controlled separate environments (biphasic) to provide host cells with oxygen and nutrients from the basal layer, while allowing a low oxygen nutrient rich environment to be developed on the apical surface (Morada et al. 2016 International Journal for Parasitology 26: 21-29).

In configuring the hollow fiber bioreactor, there are design considerations and parameters that can be varied depending upon the goals associated with expansion of the cells. One such design consideration is the size of the pores in the fiber wall. This is generally designed to allow the passage of nutrients to the cells, carry away waste, provide desired products to the cells (such as growth factors), to remove desired products from the cells, and exclude certain factors that may be present from reaching the cells. Accordingly, the pore size of the fiber walls can be varied to modify which components will pass through the walls. For example, pore size can allow the passage of large proteinaceous molecules, including growth factors, including, but not limited to, epidermal growth factor and platelet-derived growth factor. The person of ordinary skill in the art would understand how to vary the pore size depending upon the components that it is desirable to pass through the fiber walls to reach the cells or to carry material from the cells.

In some embodiments, the pore size is about 0.2 μm. In some embodiments, the pore size is about 0.1. In some embodiments, the pore size is about 0.2 μm. In some embodiments, the pore size is about 0.3 μm. In some embodiments, the pore size is about 0.4 μm. In some embodiments, the pore size is about 0.5 μm. In some embodiments, the pore size is about 0.6 μm. In some embodiments, the pore size is about 0.7 μm. In some embodiments, the pore size is about 0.8 μm. In some embodiments, the pore size is about 0.9 μm. In some embodiments, the pore size is about 1.0 μm. In some embodiments, the pore size is about 1.1 μm. In some embodiments, the pore size is about 1.2 μm. In some embodiments, the pore size is about 1.3 μm. In some embodiments, the pore size is about 1.4 μm. In some embodiments, the pore size is about 1.5 μm. In some embodiments, the pore size is about 1.6 μm. In some embodiments, the pore size is about 1.7 μm. In some embodiments, the pore size is about 1.8 μm. In some embodiments, the pore size is about 1.9 μm. In some embodiments, the pore size is about 2.0 μm. In some embodiments, the pore size is about 2.1 μm. In some embodiments, the pore size is about 2.2 μm. In some embodiments, the pore size is about 2.2 μm. In some embodiments, the pore size is about 2.3 μm. In some embodiments, the pore size is about 2.4 μm. In some embodiments, the pore size is about 2.5 μm. In some embodiments, the pore size is about 2.6 μm. In some embodiments, the pore size is about 2.7 μm. In some embodiments, the pore size is about 2.8 μm. In some embodiments, the pore size is about 2.9 μm. In some embodiments, the pore size is about 3.0 μm.

### Methods of Making Live Cell Constructs

Disclosed herein, in certain embodiments, are methods of making a live cell construct for producing a cultured milk product. In some embodiments, the method comprises (a) isolating primary mammary epithelial cells, myoepithelial cells and/or mammary progenitor cells from mammary explants from mammary tissue (e.g., breast, udder, teat tissue), biopsy sample, or raw breastmilk, to produce isolated mammary epithelial cells, myoepithelial cells and/or mammary progenitor cells; (b) culturing the isolated primary mammary epithelial cells, myoepithelial cells and/or mammary progenitor cells to produce a mixed population of primary mammary epithelial cells, mammary myoepithelial cells and mammary progenitor cells; (c) cultivating the mixed population of (b) on a scaffold having an upper surface and lower surface, to produce a polarized, monolayer of primary mammary epithelial cells, myoepithelial cells and mammary progenitor cells of the mixed population on the upper surface of the scaffold, wherein the polarized monolayer comprises an apical surface and a basal surface, thereby producing a live cell construct for producing the cultured milk product.

In some embodiments, the method comprises: a) isolating primary mammary epithelial cells, myoepithelial cells, and/or mammary progenitor cells from mammary explants from mammary tissue (e.g., breast, udder, teat tissue), biopsy sample, or raw breastmilk, to produce isolated mammary epithelial cells, myoepithelial cells, and/or mammary progenitor cells; (b) culturing the isolated primary mammary epithelial cells, myoepithelial cells, and/or mammary progenitor cells to produce a mixed population of primary mammary epithelial cells, mammary myoepithelial cells and mammary progenitor cells; (c) sorting the mixed population of primary mammary epithelial cells, myoepithelial cells, and/or mammary progenitor cells (e.g., selecting the primary mammary epithelial cells) to produce a population of primary mammary epithelial cells; and (d) cultivating the population of primary mammary epithelial on a scaffold having an upper surface and lower surface, to produce a polarized monolayer of primary mammary epithelial cells on the upper surface of the scaffold, wherein the polarized monolayer comprises an apical surface and a basal surface, thereby producing a live cell construct for producing the cultured milk product.

In some embodiments, the method comprises (a) culturing immortalized mammary epithelial cells to produce increased numbers of immortalized mammary epithelial cells; (b) cultivating the immortalized mammary epithelial cells of (a) on a scaffold, the scaffold having an upper surface and lower surface, to produce a polarized monolayer of immortalized mammary epithelial cells on the upper surface of the scaffold, wherein the polarized monolayer comprises an apical surface and a basal surface, thereby producing a live cell construct for producing the cultured milk product.

In some embodiments, the culturing and/or cultivating of the mammary cells for the live cell construct is carried out at a temperature of about 35° C. to about 39° C. (e.g., a temperature of about 35° C., 35.5° C., 36° C., 36.5° C., 37° C., 37.5° C., 38° C., 38.5° C. or about 39° C., or any value or range therein, e.g., about 35° C. to about 38° C., about 36° C. to about 39° C., about 36.5° C. to about 39° C., about 36.5° C. to about 37.5° C., or about 36.5° C. to about 38° C.). In some embodiments, the culturing and/or cultivating is carried out at a temperature of about 37° C.

In some embodiments, the culturing and/or cultivating of the mammary cells for the live cell construct is carried out at an atmospheric concentration of CO₂ of about 4% to about 6%, e.g., an atmospheric concentration of CO₂ of about 4%, 4.25%, 4.5%, 4.75%, 5%, 5.25%, 5.5%, 5.75%, or 6% or any value or range therein, e.g., about 4% to about 5.5%, about 4.5% to about 6%, about 4.5% to about 5.5%, or about 5% to about 6%). In some embodiments, the culturing and/or cultivating is carried out at an atmospheric concentration of CO₂ of about 5%.

In some embodiments, the culturing and/or cultivating of the mammary cells for the live cell construct comprises culturing and/or cultivating in a culture medium that is exchanged about every day to about every 10 days (e.g., every 1 day, every 2 days, every 3 days, every 4 days, every 5 days, every 6 days, every 7 days, every 8 days, every 9 days, every 10 days, or any value or range therein, e.g., about every day to every 3 days, about every 3 days to every 10 days, about every 2 days to every 5 days). In some embodiments, the culturing and/or cultivating further comprises culturing in a culture medium that is exchanged about every day to about every few hours to about every 10 days, e.g., about every 1, 2, 3, 4, 5, 6, 7, 8, 9, 10, 11, 12, 13, 14, 15,16, 17, 18, 19, 20, 21, 22, 23, or 24 hours to about every 1, 2, 3, 4, 5, 6, 7, 8, 9, or 10 days or any value or range therein. For example, in some embodiments, the culturing and/or cultivating further comprises culturing and/or cultivating in a culture medium that is exchanged about every 12 hours to about every 10 days, about every 10 hours to about every 5 days, or about every 5hours to about every 3 days.

In some embodiments, the live cell construct is stored in a freezer or in liquid nitrogen. The storage temperature depends on the desired storage length. For example, freezer temperature (e.g., storage at a temperature of about 0° C. to about −80° C. or less, e.g., about 0° C., −10° C., −20° C., −30° C., −40° C., −50° C., −60° C., −70° C., −80° C., −90° C., −100° C. or any value or range therein) may be used if the cells are to be used within 6 months (e.g., within 1, 2, 3, 4, 5, or 6 months). For example, liquid nitrogen may be used (e.g., storage at a temperature of −100° C. or less (e.g., about −100° C., −110° C., −120° C., −130, −140, −150, −160, −170, −180, −190° C., −200° C., or less) for longer term storage (e.g., storage of 6 months or longer, e.g., 6, 7, 8, 9, 10, 11, or 12 months, or 1, 2, 3, 4, 5, 6 or more years).

In some embodiments, the mammary cells are isolated and sorted via fluorescence-activated cell sorting, magnetic-activated cell sorting, and/or microfluidic cell sorting.

### Basal Culture Media and Lactogenic Media

In some embodiments, the culture medium comprises a carbon source, a chemical buffering system, one or more essential amino acids, one or more vitamins and/or cofactors, and one or more inorganic salts. In some embodiments, the carbon source, chemical buffering system, one or more essential amino acids, one or more vitamins and/or cofactors, and/or one or more inorganic salts are food grade.

In some embodiments, the culture medium is lactogenic culture medium. In some embodiments, the culture medium further comprises prolactin (e.g., mammalian prolactin, e.g., human prolactin), linoleic and alpha-linoleic acid, estrogen and/or progesterone. For example, in some embodiments, the culture medium comprises prolactin (or prolactin is added) in an amount from about 20 ng/ml to about 200 ng/L of culture medium, e.g., about 20, 30, 40, 50, 60, 70, 80,90, 100, 110, 120, 130, 140, 150, 160, 170, 180, 190, or 200 ng/ml or any value or range therein. In some embodiments, the culture medium comprises prolactin (or prolactin is added) in an amount from about 20 ng/ml to about 195 ng/ml, about 50 ng/ml to about 150 ng/ml, about 25 ng/mL to about 175 ng/ml, about 45 ng/mL to about 200 ng/mL, or about 75 ng/ml to about 190 ng/ml of culture medium. In some embodiments, the culture medium further comprises other factors to improve efficiency, including, but not limited to, insulin, an epidermal growth factor, and/or a hydrocortisone.

In some embodiments, the culture medium comprises a carbon source in an amount from about 1 g/L to about 15 g/L of culture medium (e.g., about 1, 2, 3, 4, 5, 6, 7, 8, 9, 10, 11, 12, 13, 14 or 15 g/L or any value or range therein), or about 1, 2, 3, 4, 5 or 6 g/L to about 7, 8, 9, or 10, 11, 12, 13, 14 or 15 g/L of the culture medium. Non-limiting examples of a carbon source include glucose and/or pyruvate. For example, in some embodiments, the culture medium comprises glucose in an amount from about 1 g/L to about 12 g/L of culture medium, e.g., about 1, 2, 3, 4, 5, 6, 7, 8, 9, 10, 11, or 12 g/L or any value or range therein. In some embodiments, the culture medium comprises glucose in an amount from about 1 g/L to about 6 g/L, about 4 g/L to about 12 g/L, about 2.5 g/L to about 10.5 g/L, about 1.5 g/L to about 11.5 g/L, or about 2 g/L to about 10 g/L of culture medium. In some embodiments, the culture medium comprises glucose in an amount from about 1, 2, 3, or 4 g/L to about 5, 6, 7, 8, 9, 10, 11, or 12 g/L or about 1, 2, 3, 4, 5, or 6 g/L to about 7, 8, 9, 10, 11, or 12 g/L. In some embodiments, the culture medium comprises pyruvate in an amount from about 5 g/L to about 15 g/L of culture medium, e.g., about 5, 6, 7, 8, 9, 10, 11, 12, 13, 14, or 15 g/L or any value or range therein. In some embodiments, the culture medium comprises pyruvate in an amount from about 5 g/L to about 14.5 g/L, about 10 g/L to about 15 g/L, about 7.5 g/L to about 10.5 g/L, about 5.5 g/L to about 14.5 g/L, or about 8 g/L to about 10 g/L of culture medium. In some embodiments, the culture medium comprises pyruvate in an amount from about 5, 6, 7, or 8 g/L to about 9, 10, 11, 12, 13, 14 or 15 g/L or about 5, 6, 7, 8, 9, or 10 g/L to about 11, 12, 13, 14 or 15 g/L.

In some embodiments, the culture medium comprises a chemical buffering system in an amount from about 1 g/L to about 4 g/L (e.g., about 1, 1.5, 2, 2.5, 3, 3.5, or 4 g/L or any value or range therein) of culture medium or about 10 mM to about 25 mM (e.g., about 10, 11, 12, 13, 14, 15, 16, 17, 18, 19, 20, 21, 22, 23, 24, or 25 mM or any value or range therein). In some embodiments, the chemical buffering system includes, but is not limited to, sodium bicarbonate and/or 4-(2-hydroxyethyl)-1-piperazineethanesulfonic acid (HEPES). For example, in some embodiments, the culture medium comprises sodium bicarbonate in an amount from about 1 g/L to about 4 g/L of culture medium, e.g., about 1, 1.5, 2, 2.5, 3, 3.5, or 4 g/L or any value or range therein. In some embodiments, the culture medium comprises sodium bicarbonate in an amount from about 1 g/L to about 3.75 g/L, about 1.25 g/L to about 4 g/L, about 2.5 g/L to about 3 g/L, about 1.5 g/L to about 4 g/L, or about 2 g/L to about 3.5 g/L of culture medium. In some embodiments, the culture medium comprises HEPES in an amount from about 10 mM to about 25 mM, e.g., about 10, 11, 12, 13, 14, 15, 16, 17, 18, 19, 20, 21, 22, 23, 24, or 25 mM or any value or range therein. In some embodiments, the culture medium comprises HEPES in an amount from about 11 mM to about 25 mM, about 10 mM to about 20 mM, about 12.5 mM to about 22.5 mM, about 15 mM to about 20.75 mM, or about 10 mM to about 20 mM.

In some embodiments, the culture medium comprises one or more essential amino acids in an amount from about 0.5 mM to about 5 mM (e.g., about 0.5, 1, 1.5, 2, 2.5, 3, 3.5, 4, 4.5, or 5 mM or any value or range therein) or about 0.5, 1, 1.5, 2 mM to about 2.5, 3, 3.5, 4, 4.5, or 5 mM. In some embodiments, the one or more essential amino acids is histidine, isoleucine, leucine, lysine, methionine, phenylalanine, threonine, tryptophan, valine, and/or arginine. For example, in some embodiments, the culture medium comprises arginine in an amount from about 0.5 mM to about 5 mM, e.g., about 0.5, 1, 1.5, 2, 2.5, 3, 3.5, 4, 4.5, or 5 mM or any value or range therein. In some embodiments, the culture medium comprises an essential amino acids in an amount from about 0.5 mM to about 4.75 mM, about 2 mM to about 3.5 mM, about 0.5 mM to about 3.5 mM, about 1 mM to about 5 mM, or about 3.5 mM to about 5 mM.

In some embodiments, the culture medium comprises one or more vitamins and/or cofactors in an amount from about 0.01 μM to about 50 μM (e.g., about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 3, 4, 5, 6, 7,8,9, 10, 12.5, 15, 17.5,20, 25, 30, 35, 40, 45, 46, 47, 48, 49, 49.025, 49.05, 49.075, or 50 μM or any value or range therein) or about 0.01, 0.02, 0.03, 0.04, 0.05, 0.06, 0.07, 0.08, 0.09, 0.1, 0.2, 0.3, 0.4, 0.5, 0.6, 0.7, 0.8, or 0.9 μM to about 1, 1.1, 1.2, 1.3, 1.4, 1.5, 1.6, 1.7, 1.8, 1.9, 2, 2.1, 2.2, 2.3, 2.4, 2.5, 3, 4, 5, 6 μM or about 0.02, 0.025, 0.05, 0.075, 1, 1.5, 2, 3, 4, 5, 6, 7, 8, 9, 10 μM to about 12.5, 15, 17.5, 20, 25, 30, 35, 40, 45, 46, 47, 48, 49, 49.025, 49.05, 49.075, or 50 μM. In some embodiments, one or more vitamins and/or cofactors include, but are not limited to, thiamine and/or riboflavin. For example, in some embodiments, the culture medium comprises thiamine in an amount from about 0.025 μM to about 50 μM, e.g., about 0.025, 0.05, 0.075, 1, 1.5, 2, 3, 4, 5, 6, 7, 8, 9, 10, 12.5, 15, 17.5, 20, 25,30, 35, 40, 45, 46, 47, 48, 49, 49.025, 49.05, 49.075, or 50 μM or any value or range therein. In some embodiments, the culture medium comprises thiamine in an amount from about 0.025 μM to about 45.075 μM, about 1 μM to about 40 μM, about 5 μM to about 35.075 μM, about 10 μM to about 50 μM, or about 0.05 μM to about 45.5 μM. In some embodiments, the culture medium comprises riboflavin in an amount from about 0.01 μM to about 3 μM, e.g., about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 μM or any value or range therein. In some embodiments, the culture medium comprises riboflavin in an amount from about 0.01 μM to about 2.05 μM, about 1 μM to about 2.95 μM, about 0.05 μM to about 3 μM, about 0.08 μM to about 1.55 μM, or about 0.05 μM to about 2.9 μM.

In some embodiments, the culture medium comprises one or more inorganic salts in an amount from about 100 mg/L to about 150 mg/L of culture medium (e.g., about 100, 105, 110, 115, 120, 125, 130, 135, 140, 145, or 150 mg/L or any value or range therein) or about 100 mg/L to about 150 mg/L of culture medium (e.g., about 100, 105, 110, 115, 120, 125, 130, 135, 140, 145, or 150 mg/L or any value or range therein). In some embodiments, one or more inorganic salts include, but are not limited to, calcium and/or magnesium. For example, in some embodiments, the culture medium comprises calcium in an amount from about 100 mg/L to about 150 mg/L of culture medium, e.g., about 100, 105, 110, 115, 120, 125, 130, 135, 140, 145, or 150 mg/L or any value or range therein. In some embodiments, the culture medium comprises arginine in an amount from about 100 mg/L to about 125 mg/L, about 105 mg/L to about 150 mg/L, about 120 mg/L to about 130 mg/L, or about 100 mg/L to about 145 mg/L of culture medium. In some embodiments, the culture medium comprises magnesium in an amount from about 0.01 mM to about 1 mM, e.g., about 0.01,0.02, 0.03, 0.04, 0.05, 0.06, 0.07, 0.08, 0.09, 0.1, 0.2, 0.3, 0.4, 0.5, 0.6, 0.7, 0.8, 0.9, 0.91, 0.92, 0.93, 0.94, 0.95, 0.96, 0.97, 0.98, 0.99, or 1 mM or any value or range therein. In some embodiments, the culture medium comprises magnesium in an amount from about 0.05 mM to about 1 mM, about 0.01 mM to about 0.78 mM, about 0.5 mM to about 1 mM, about 0.03 mM to about 0.75 mM, or about 0.25 mM to about 0.95 mM.

In some embodiments, the culture medium comprises a carbon source in an amount from about 1 g/L to about 15 g/L of culture medium (e.g., about 1, 2, 3, 4, 5, 6, 7, 8, 9, 10, 11, 12, 13, 14 or 15 g/L or any value or range therein), or about 1, 2, 3, 4, 5 or 6 g/L to about 7, 8, 9, or 10, 11, 12, 13, 14 or 15 g/L of the culture medium. In some embodiments, the carbon source includes, but is not limited to, glucose and/or pyruvate. For example, in some embodiments, the culture medium comprises glucose in an amount from about 1 g/L to about 12 g/L of culture medium, e.g., about 1, 2, 3, 4, 5, 6, 7, 8, 9, 10, 11, or 12 g/L or any value or range therein. In some embodiments, the culture medium comprises glucose in an amount from about 1 g/L to about 6 g/L, about 4 g/L to about 12 g/L, about 2.5 g/L to about 10.5 g/L, about 1.5 g/L to about 11.5 g/L, or about 2 g/L to about 10 g/L of culture medium. In some embodiments, the culture medium comprises pyruvate at an amount of about 5 g/L to about 15 g/L of culture medium, e.g., about 5, 6, 7, 8, 9, 10, 11, 12, 13, 14, or 15 g/L or any value or range therein. In some embodiments, the culture medium comprises pyruvate in an amount from about 5 g/L to about 14.5 g/L, about 10 g/L to about 15 g/L, about 7.5 g/L to about 10.5 g/L, about 5.5 g/L to about 14.5 g/L, or about 8 g/L to about 10 g/L of culture medium.

In some embodiments, the culture medium comprises a chemical buffering system in an amount from about 1 g/L to about 4 g/L (e.g., about 1, 1.5, 2, 2.5, 3, 3.5, or 4 g/L or any value or range therein) of culture medium or about 10 mM to about 25 mM (e.g., about 10, 11, 12, 13, 14, 15, 16, 17, 18, 19, 20, 21, 22, 23, 24, or 25 mM or any value or range therein). In some embodiments, the chemical buffering system includes, but is not limited to, sodium bicarbonate and/or HEPES. For example, in some embodiments, the culture medium comprises sodium bicarbonate in an amount from about 1 g/L to about 4 g/L of culture medium, e.g., about 1, 1.5, 2, 2.5, 3, 3.5, or 4 g/L or any value or range therein. In some embodiments, the culture medium comprises sodium bicarbonate in an amount from about 1 g/L to about 3.75 g/L, about 1.25 g/L to about 4 g/L, about 2.5 g/L to about 3 g/L, about 1.5 g/L to about 4 g/L, or about 2 g/L to about 3.5 g/L of culture medium. In some embodiments, the culture medium comprises HEPES in an amount from about 10 mM to about 25 mM, e.g., about 10, 11, 12, 13, 14, 15, 16, 17, 18, 19, 20, 21, 22, 23, 24, or 25 mM or any value or range therein. In some embodiments, the culture medium comprises HEPES in an amount from about 1 mM to about 25 mM, about 10 mM to about 20 mM, about 12.5 mM to about 22.5 mM, about 15 mM to about 20.75 mM, or about 10 mM to about 20 mM.

In some embodiments, the culture medium comprises one or more essential amino acids in an amount from about 0.5 mM to about 5 mM (e.g., about 0.5, 1, 1.5, 2, 2.5, 3, 3.5, 4, 4.5, or 5 mM or any value or range therein) or about 0.5, 1, 1.5, 2 mM to about 2.5, 3, 3.5, 4, 4.5, or 5 mM. In some embodiments, one or more essential amino acids is arginine and/or cysteine. For example, in some embodiments, the culture medium comprises arginine in an amount from about 0.5 mM to about 5 mM, e.g., about 0.5, 1, 1.5, 2, 2.5, 3, 3.5, 4, 4.5, or 5 mM or any value or range therein. In some embodiments, the culture medium comprises arginine in an amount from about 0.5 mM to about 4.75 mM, about 2 mM to about 3.5 mM, about 0.5 mM to about 3.5 mM, about 1 mM to about 5 mM, or about 3.5 mM to about 5 mM. For example, in some embodiments, the culture medium comprises cysteine in an amount from about 0.5 mM to about 5 mM, e.g., about 0.5, 1, 1.5, 2, 2.5, 3, 3.5, 4, 4.5, or 5 mM or any value or range therein. In some embodiments, the culture medium comprises cysteine in an amount from about 0.5 mM to about 4,75 mM, about 2 mM to about 3.5 mM, about 0.5 mM to about 3.5 mM, about 1 mM to about 5 mM, or about 3.5 mM to about 5 mM.

In some embodiments, the culture medium comprises one or more vitamins and/or cofactors in an amount from about 0.01 μM to about 50 μM (e.g., about 0.01, 0.02, 0.03, 0.04, 0.05, 0.06, 0.07, 0.08, 0.09, 0.1,0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 3, 4, 5, 6, 7, 8,9, 10, 12.5, 15, 17.5, 20, 25, 30, 35, 40, 45, 46, 47, 48, 49, 49.025, 49.05, 49.075, or 50 μM or any value or range therein) or about 0.01, 0.02, 0.03, 0.04, 0.05, 0.06, 0.07, 0.08, 0.09, 0.1, 0.2, 0.3, 0.4, 0.5, 0.6, 0.7, 0.8, or 0.9 μM to about 1, 1.1, 1.2, 1.3, 1.4, 1.5, 1.6, 1.7, 1.8, 1.9, 2, 2.1, 2.2, 2.3, 2.4, 2.5, 3, 4, 5, 6 μM or about 0.02, 0.025, 0.05, 0.075, 1, 1.5, 2, 3, 4, 5, 6, 7, 8, 9, 10 μM to about 12.5, 15, 17.5, 20, 25, 30, 35, 40, 45, 46, 47, 48, 49, 49.025, 49.05, 49.075, or 50 μM. In some embodiments, one or more vitamins and/or cofactors includes, but is not limited to, thiamine and/or riboflavin. For example, in some embodiments, the culture medium comprises thiamine in an amount from about 0.025 μM to about 50 μM, e.g., 0.025, 0.05, 0.075, 1, 1.5, 2, 3, 4, 5, 6, 7, 8, 9, 10, 12.5, 15, 17.5, 20, 25, 30, 35, 40, 45, 46, 47, 48, 49, 49.025, 49.05, 49.075, or 50 μM or any value or range therein. In some embodiments, the culture medium comprises thiamine in an amount from about 0.025 μM to about 45.075 μM, about 1 μM to about 40 μM, about 5 μM to about 35.075 μM, about 10 μM to about 50 μM, or about 0.05 μM to about 45.5 μM. In some embodiments, the culture medium comprises riboflavin in an amount from about 0.01 μM to about 3 μM, e.g., 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 μM or any value or range therein. In some embodiments, the culture medium comprises riboflavin in an amount from about 0.01 μM to about 2.05 μM, about 1 μM to about 2.95 μM, about 0.05 μM to about 3 μM, about 0.08 μM to about 1.55 μM, or about 0.05 μM to about 2.9 μM.

In some embodiments, the culture medium comprises one or more inorganic salts in an amount from about 100 mg/L to about 150 mg/L of culture medium (e.g., about 100, 105, 110, 115, 120, 125, 130, 135, 140, 145, or 150 mg/L or any value or range therein) or about 100 mg/L to about 150 mg/L of culture medium (e.g., about 100, 105, 110, 115, 120, 125, 130, 135, 140, 145, or 150 mg/L or any value or range therein). In some embodiments, exemplary one or more inorganic salts is calcium and/or magnesium. For example, in some embodiments, the culture medium comprises calcium in an amount from about 100 mg/L to about 150 mg/L of culture medium, e.g., about 100, 105, 110, 115, 120, 125, 130, 135, 140, 145, or 150 mg/L or any value or range therein. In some embodiments, the culture medium comprises arginine in an amount from about 100 mg/L to about 125 mg/L, about 105 mg/L to about 150 mg/L, about 120 mg/L to about 130 mg/L, or about 100 mg/L to about 145 mg/L of culture medium. In some embodiments, the culture medium comprises magnesium in an amount from about 0.01 mM to about 1 mM, e.g., about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 0.91, 0.92, 0.93, 0.94, 0.95, 0.96, 0.97, 0.98, 0.99, or 1 mM or any value or range therein. In some embodiments, the culture medium comprises magnesium in an amount from about 0.05 mM to about 1 mM, about 0.01 mM to about 0.78 mM, about 0.5 mM to about 1 mM, about 0.03 mM to about 0.75 mM, or about 0.25 mM to about 0.95 mM.

In some embodiments, the carbon source, chemical buffering system, one or more essential amino acids, one or more vitamins and/or cofactors, and/or one or more inorganic salts is food grade.

In some embodiments, the culture medium is lactogenic culture medium, e.g., the culture medium further comprises prolactin (e.g., mammalian prolactin, e.g., human prolactin). For example, in some embodiments, the culture medium comprises prolactin (or prolactin is added) in an amount from about 20 ng/mL to about 200 ng/L of culture medium, e.g., about 20, 30, 40, 50, 60, 70, 80, 90, 100, 110, 120, 130, 140, 150, 160, 170, 180, 190, or 200 ng/mL or any value or range therein. In some embodiments, the culture medium comprises prolactin (or prolactin is added) in an amount from about 20 ng/ml to about 195 ng/ml, about 50 ng/ml to about 150 ng/ml, about 25 ng/ml to about 175 ng/ml, about 45 ng/ml to about 200 ng/ml, or about 75 ng/mL to about 190 ng/mL of culture medium. In some embodiments, the methods further comprise adding prolactin to the culture medium, thereby providing a lactogenic culture medium. In some embodiments, the prolactin is produced by a microbial cell and/or a human cell expressing a recombinant prolactin (e.g., a prolactin comprising a substitution of a serine residue at position 179 of the prolactin gene with aspartate (S179D), e.g., S179D-prolactin). In some embodiments, adding prolactin to the culture medium comprises conditioning culture medium by culturing cells that express and secrete prolactin, and applying the conditioned culture medium comprising prolactin to the basal surface of the monolayer of primary mammary epithelial cells, the basal surface of the monolayer of the mixed population, or the basal surface of the monolayer of live immortalized mammary epithelial cells.

In some embodiments, the culture medium further comprises other factors to improve efficiency, including, but not limited to, insulin, an epidermal growth factor, and/or a hydrocortisone. In some embodiments, the methods of the present invention further comprise adding other factors (e.g., insulin, an epidermal growth factor, and/or a hydrocortisone) to the culture medium, e.g., to improve efficiency.

### Methods of Producing Cultured Milk Products

Disclosed herein, in certain embodiments, are methods of making a cultured milk product. In some embodiments, the method comprises culturing a live cell construct disclosed herein in a bioreactor comprising a basal compartment and an apical compartment, wherein the basal compartment comprises a culture media and the mammary cells secret the cultured milk product into the apical compartment.

In some embodiments, the live cell construct comprises a scaffold comprising an upper surface and a lower surface and a polarized monolayer of live primary mammary epithelial cells, a continuous polarized monolayer of a mixed population of live primary mammary epithelial cells, mammary myoepithelial cells and mammary progenitor cells, and/or a continuous polarized monolayer of live immortalized mammary epithelial cells having an apical surface and a basal surface, wherein the continuous polarized monolayer of live primary mammary epithelial cells, the continuous polarized monolayer of the mixed population of live primary mammary epithelial cells, mammary myoepithelial cells and mammary progenitor cells and/or the continuous polarized monolayer of live immortalized mammary epithelial cells are located on the upper surface of scaffold

In some embodiments, the lower surface of the scaffold is adjacent to the basal compartment. In some embodiments, the apical surface of the continuous polarized monolayer of live primary mammary epithelial cells, the continuous polarized monolayer of the mixed population of live primary mammary epithelial cells, mammary myoepithelial cells and mammary progenitor cells, and/or the continuous polarized monolayer of live immortalized mammary epithelial cells is adjacent to the apical compartment. In some embodiments, the continuous polarized monolayer of live primary epithelial mammary cells, the live primary epithelial mammary cells of the continuous polarized monolayer of the mixed population of live primary mammary epithelial cells, mammary myoepithelial cells and mammary progenitor cells, or the continuous polarized monolayer of immortalized mammary epithelial cells secretes milk through its apical surface into the apical compartment, thereby producing milk in culture.

In some embodiments, the polarized monolayer of epithelial mammary cells forms a barrier that divides the apical compartment and the basal compartment, wherein the basal surface of the mammary cells are attached to the scaffold and the apical surface is oriented toward the apical compartment.

In some embodiments, the basal compartment is adjacent to the lower surface of the scaffold. In some embodiments, the basal compartment comprises a culture medium in fluidic contact with the basal surface of the polarized monolayer of mammary epithelial cells (e.g., the polarized monolayer of primary mammary epithelial cells, the polarized the monolayer of the mixed population, or the polarized monolayer of live immortalized mammary epithelial cells).

In some embodiments, the culture medium comprises a carbon source, a chemical buffering system, one or more essential amino acids, one or more vitamins and/or cofactors, and one or more inorganic salts.

In some embodiments, the bioreactor comprises an apical compartment that is adjacent to the apical surface of the monolayer. In some embodiments, the apical compartment is adjacent to the upper surface of the scaffold.

In some embodiments, the total cell density of mammary cells in the bioreactor is at least 10¹¹ mammary cells. In some embodiments, the total cell density of mammary cells in the bioreactor is at least 10¹² mammary cells. In some embodiments, the total cell density of mammary cells in the bioreactor is at least 10¹³ mammary cells.

In some embodiments, the total cell density of mammary cells in the bioreactor is about 20 to 55 cells per 100 μm². In some embodiments, the total cell density of mammary cells in the bioreactor is about 20 cells per 100 μm². In some embodiments the total cell density of mammary cells in the bioreactor is 25 cells per 100 μm². In some embodiments, the total cell density of mammary cells in the bioreactor is about 30 cells per 100 μm². In some embodiments, the total cell density of mammary cells in the bioreactor is about 35 cells per 100 μm². In some embodiments, the total cell density of mammary cells in the bioreactor is about 40 cells per 100 μm². In some embodiments, the total cell density of mammary cells in the bioreactor is about 45 cells per 100 μm². In some embodiments, the total cell density of mammary cells in the bioreactor is about 50 cells per 100 μm². In some embodiments, the total cell density of mammary cells in the bioreactor is about 55 cells per 100 μm².

In some embodiments, the total surface area of mammary cells within the bioreactor is at least about 1.5 m². In some embodiments, the total surface area of mammary cells within the bioreactor is at least about 2 m². In some embodiments, the total surface area of mammary cells within the bioreactor is at least about 2.5 m². In some embodiments, the total surface area of mammary cells within the bioreactor is at least about 3 m². In some embodiments, the total surface area of mammary cells within the bioreactor is at least about 4 m². In some embodiments, the total surface area of mammary cells within the bioreactor is at least about 5 m². In some embodiments, the total surface area of mammary cells within the bioreactor is at least about 10 m². In some embodiments, the total surface area of mammary cells within the bioreactor is at least about 15 m². In some embodiments, the total surface area of mammary cells within the bioreactor is at least about 20 m². In some embodiments, the total surface area of mammary cells within the bioreactor is at least about 25 m². In some embodiments, the total surface area of mammary cells within the bioreactor is at least about 50 m². In some embodiments, the total surface area of mammary cells within the bioreactor is at least about 100 m². In some embodiments, the total surface area of mammary cells within the bioreactor is at least about 250 m². In some embodiments, the total surface area of mammary cells within the bioreactor is at least about 500 m².

In some embodiments, the bioreactor maintains a temperature of about 27° C. to about 39° C. (e.g., a temperature of about 27° C., 28° C., 29° C., 30° C., 31° C., 32° C., 33° C., 34° C., 35° C., 35° C., 35.5° C., 36° C., 36.5° C., 37° C., 37.5° C., 38° C., 38.5° C. or about 39° C., or any value or range therein, e.g., about 27° C. to about 38° C., about 36° C. to about 39° C., about 36.5° C. to about 39° C., about 36.5° C. to about 37.5° C., or about 36.5° C. to about 38° C.). In some embodiments, the bioreactor maintains a temperature of about 37° C.

In some embodiments, the bioreactor has an atmospheric concentration of CO₂ of about 4% to about 6%, e.g., an atmospheric concentration of CO₂ of about 4%, 4.25%, 4.5%, 4.75%, 5%, 5.25%, 5.5%, 5.75%, or 6% or any value or range therein, e.g., about 4% to about 5.5%, about 4.5% to about 6%, about 4.5% to about 5.5%, or about 5% to about 6%). In some embodiments, the bioreactor has an atmospheric concentration of CO₂ of about 5%.

In some embodiments, the bioreactor has an atmospheric concentration of CO₂ of about 4% to about 6%, e.g., an atmospheric concentration of CO₂ of about 4%, 4.25%, 4.5%, 4.75%, 5%, 5.25%, 5.5%, 5.75%, or 6% or any value or range therein, e.g., about 4% to about 5.5%, about 4.5% to about 6%, about 4.5% to about 5.5%, or about 5% to about 6%). In some embodiments, the bioreactor has an atmospheric concentration of CO₂ of about 5%.

In some embodiments, the method comprises monitoring the concentration of dissolved O₂ and CO₂. In some embodiments, the concentration of dissolved O₂ is maintained between about 10% to about 25% or any value or range therein (e.g., about 10, 11, 12, 13, 14, 15, 16, 17, 18, 19, 20, 21, 22, 23, 24, or 25%). For example, in some embodiments, the concentration of dissolved O₂ is maintained between about 12% to about 25%, about 15% to about 22%, about 10% to about 20%, about 15%, about 20%, or about 22%. In some embodiments, the concentration of CO₂ is maintained between about 4% to about 6%, e.g., a concentration of CO₂ of about 4%, 4.25%, 4.5%, 4.75%, 5%, 5.25%, 5.5%, 5.75%, or 6% or any value or range therein, e.g., about 4% to about 5.5%, about 4.5% to about 6%, about 4.5% to about 5.5%, or about 5% to about 6%). In some embodiments, the concentration of CO₂ is maintained at about 5%.

In some embodiments, the culture medium is exchanged about every day to about every 10 days (e.g., every 1 day, every 2 days, every 3 days, every 4 days, every 5 days, every 6 days, every 7 days, every 8 days, every 9 days, every 10 days, or any value or range therein, e.g., about every day to every 3 days, about every 3 days to every 10 days, about every 2 days to every 5 days). In some embodiments, the culture medium is exchanged about every day to about every few hours to about every 10 days, e.g., about every 1, 2, 3, 4, 5, 6, 7, 8, 9, 10, 11, 12, 13, 14, 15, 16, 17, 18, 19, 20, 21, 22, 23, or 24 hours to about every 1, 2, 3, 4, 5, 6, 7, 8, 9, or 10 days or any value or range therein. For example, in some embodiments, the culture medium is exchanged about every 12 hours to about every 10 days, about every 10 hours to about every 5 days, or about every 5 hours to about every 3 days.

In some embodiments, the method comprises monitoring the glucose concentration and/or rate of glucose consumption in the culture medium and/or in the lactogenic culture medium. In some embodiments, the prolactin is added when the rate of glucose consumption in the culture medium is steady state.

In some embodiments, the method further comprises applying transepithelial electrical resistance (TEER) to measure the maintenance of the monolayer of epithelial cells. TEER measures a voltage difference between the fluids (e.g., media) in two compartments (e.g., between the apical and basal compartments), wherein if the barrier between the compartments loses integrity, the fluids in the two compartments may mix. When there is fluid mixing, the voltage difference will be reduced or eliminated; a voltage difference indicates that the barrier is intact. In some embodiments, upon detection of a loss of voltage by TEER, a scaffold (e.g., a Transwell® filter, a microstructured bioreactor, a decellularized tissue, a hollow fiber bioreactor, etc.) is reinoculated with additional cells and allowed time to reestablish a barrier (e.g., a monolayer) before resuming production of the cultured milk product (e.g., milk production).

In some embodiments, the method further comprises collecting the cultured milk product from the apical compartment to produce collected cultured milk product. In some embodiments, the collecting is via a port, via gravity, and/or via a vacuum. In some embodiments, a vacuum is attached to a port.

In some embodiments, the method further comprises freezing the collected cultured milk product to produce frozen cultured milk product and/or lyophilizing the collected cultured milk product to produce lyophilized cultured milk product.

In some embodiments, the method further comprises packaging the collected cultured milk product, the frozen cultured milk product and/or the lyophilized cultured milk product into a container.

In some embodiments, the method further comprises extracting one or more components from the collected cultured milk product. Non-limiting examples of components from the collected cultured milk product include milk protein, lipid, carbohydrate, vitamin, and/or mineral contents. In some embodiments, the components from the collected cultured milk product are lyophilized and/or concentrated to produce a lyophilized or a concentrated cultured milk product component product. In some embodiments, the components from the collected cultured milk product are concentrated by, e.g., membrane filtration and/or reverse osmosis. In some embodiments, the lyophilized or concentrated cultured milk product component product is packaged in a container, optionally wherein the container is sterile and/or a food grade container. In some embodiments, the container is vacuum-sealed. In some embodiments, the container is a canister, ajar, a bottle, a bag, a box, or a pouch.

### Cultured Milk Products

Disclosed herein, in certain embodiments, are cultured milk products. In some embodiments, the cultured milk product is a standardized, sterile cultured milk product. In some embodiments, the cultured milk product is for nutritional use.

In some embodiments, the cultured milk product is produced by any method disclosed herein.

Breast milk contains low but measurable concentrations of environmental contaminants, health-harming chemicals from industry and manufacturing products that are widely spread in the environment. Environmental contaminants are partly secreted in breast milk. The contaminant levels in breast milk reflect those in the mother's body and are therefore ideal for monitoring exposure levels. Toxic environmental contaminants can be transferred from mother to infant via breastfeeding. Persistent organic pollutants (POPs) are a family of lipophilic stable chemicals that bioaccumulate in adipose tissue and create a lasting toxic body burden. Breastfeeding provides a significant source of exposure to POPs early in human life, the effects of which are unknown.

In some embodiments, the cultured milk product does not comprise or is substantially free of one or more environmental contaminants. In some embodiments, the cultured milk product does not comprise or is substantially free of persistent organic pollutants (POPs). In some embodiments, the cultured milk product does not comprise or is substantially free of polychlorinated dibenzo-p-dioxins (PCDDs), polychlorinated dibenzofurans (PCDFs), polychlorinated biphenyls (PCBs) and pesticides such as DDT.

Heavy metals such as mercury, lead, arsenic, cadmium, nickel, chromium, cobalt, zinc, and other potentially toxic metals that are dispersed throughout the environment also have bioaccumulative features known to accumulate in human milk and thus are of concern to the nursing infant. Metal in breast milk originates from exogenous sources, i.e., uptake via contaminated air, food, and drinking water, and endogenous release along with essential trace elements. For example, lead and mercury are equally dispersed in the human food chain, and their impact on fetal development is heavily determined by the mother's diet and nutritional status. The exposures to toxic metals have significant public health implication, even at small concentrations and acute exposures, these metals remain toxic to humans. A nursing infant may be exposed to toxic metals in a period of highest susceptibility. Nursing infants may be exposed to heavy metals through breast milk in excess of what they should, and exposure may have health implication for the infants. For infants in particular, these exposures may have adverse effect on the developing central nervous system, leaving a life-long defect on their cognitive abilities.

In some embodiments, the cultured milk product does not comprise or is substantially free of one or more heavy metals, such as arsenic, lead, cadmium, nickel, mercury, chromium, cobalt, and zinc. In some embodiments, the cultured milk product does not comprise or is substantially free of arsenic. In some embodiments, the cultured milk product does not comprise or is substantially free of lead. In some embodiments, the cultured milk product does not comprise or is substantially free of cadmium. In some embodiments, the cultured milk product does not comprise or is substantially free of nickel. In some embodiments, the cultured milk product does not comprise or is substantially free of mercury. In some embodiments, the cultured milk product does not comprise or is substantially free of chromium. In some embodiments, the cultured milk product does not comprise or is substantially free of cobalt. In some embodiments, the cultured milk product does not comprise or is substantially free of zinc. In some embodiments, the cultured milk product does not comprise or is substantially free of arsenic, lead, cadmium, nickel, mercury, chromium, cobalt, and zinc.

Foreign allergenic proteins can be difficult to distinguish from endogenous human milk proteins. Food proteins with allergenic potential that have been detected in human milk include hen's egg and peanut proteins. There are eight major food allergens, known as the big 8, that are responsible for most of the serious food allergy reactions in the U.S. The big 8 list is comprised of milk, egg, fish, crustacean shellfish, tree nuts, peanuts, wheat, and soybean allergens. Proteins known to cause egg allergy include ovomucoid, ovalbumin, and conalbumin. Peanuts proteins include arachin 6, arachin 3, conarachin, main allergen Arah1, and arachin Arah2. As an example of maternal dietary protein transportation to milk, it has been shown that the consumption of one egg per day leads to higher concentrations of the chicken egg allergen ovalbumin (OVA) in human milk compared to egg-avoiding mothers.

In some embodiments, the cultured milk product does not comprise or is substantially free of one or more food allergens. In some embodiments, the cultured milk product does not comprise or is substantially free of egg, fish, crustacean shellfish, tree nuts, peanuts, wheat, and soybean allergens. In some embodiments, the cultured milk product does not comprise or is substantially free of egg allergens. In some embodiments, the cultured milk product does not comprise or is substantially free of fish allergens. In some embodiments, the cultured milk product does not comprise or is substantially free of crustacean allergens. In some embodiments, the cultured milk product does not comprise or is substantially free of tree nut allergens. In some embodiments, the cultured milk product does not comprise or is substantially free of peanut allergens. In some embodiments, the cultured milk product does not comprise or is substantially free of wheat allergens. In some embodiments, the cultured milk product does not comprise or is substantially free of soybean allergens.

In some embodiments, the cultured milk product does not comprise or is substantially free of arachin 6, arachin 3, conarachin, Arah1, and Arah2.

In some embodiments, the cultured milk product does not comprise or is substantially free of ovalbumin (OVA).

Having described the present invention, the same will be explained in greater detail in the following examples, which are included herein for illustration purposes only, and which are not intended to be limiting to the invention.

### EXAMPLES

### Example 1

A cell culture system designed for the collection of milk should support compartmentalized secretion of the product such that the milk is not exposed to the media that provides nutrients to the cells. In the body, milk-producing epithelial cells line the interior surface of the mammary gland as a continuous monolayer. The monolayer is oriented such that the basal surface is attached to an underlying basement membrane, while milk is secreted from the apical surface and stored in the luminal compartment of the gland, or alveolus, until it is removed during milking or feeding. Tight junctions along the lateral surfaces of the cells ensure a barrier between the underlying tissues and the milk located in the alveolar compartment. Therefore, in vivo, the tissue of the mammary gland is arranged such that milk secretion is compartmentalized, with the mammary epithelial cells themselves establishing the interface and maintaining the directional absorption of nutrients and secretion of milk.

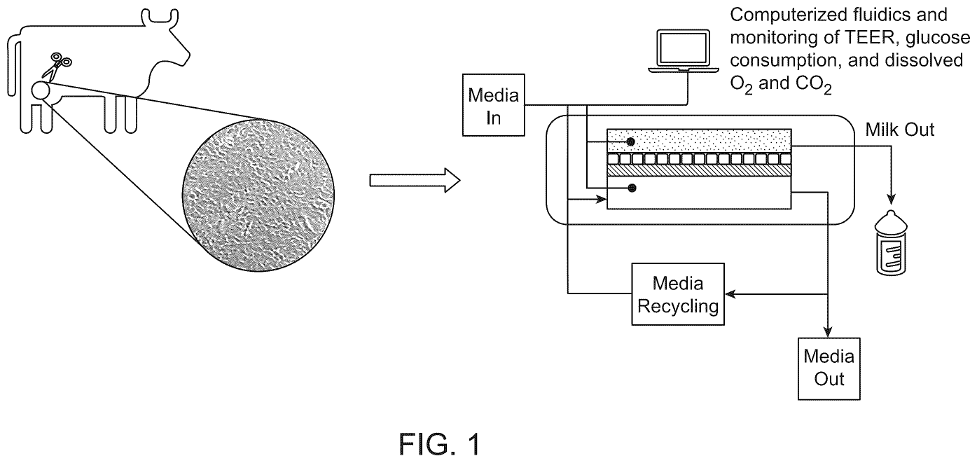

The present disclosure describes a cell culture apparatus that recapitulates the compartmentalizing capability of the mammary gland that is used to collect milk from mammary epithelial cells grown outside of the body. Such an apparatus can include a scaffold to support the proliferation of mammary cells at the interface between two compartments, such that the epithelial monolayer provides a physical boundary between the nutrient medium and the secreted milk. In addition to providing a surface for growth, the scaffold provides spatial cues that guide the polarization of the cells and ensures the directionality of absorption and secretion. This invention describes the preparation, cultivation, and stimulation of mammary epithelial cells in a compartmentalizing cell culture apparatus for the production and collection of milk for nutritional use (see e.g., FIG. 1).

Preparation of mammary epithelial cells. Mammary epithelial cells are obtained from surgical explants of dissected mammary tissue (e.g., breast, udder, teat), biopsy sample, or raw breastmilk. Generally, after surgical dissection of the mammary tissue, any fatty or stromal tissue is manually removed under aseptic conditions, and the remaining tissue of the mammary gland is enzymatically digested with collagenase and/or hyaluronidase prepared in a chemically defined nutrient media, which should be composed of ingredients that are “generally recognized as safe” (GRAS). The sample is maintained at 37° C. with gentle agitation. After digestion, a suspension of single cells or organoids is collected, either by centrifugation or by pouring the sample through a sterile nylon cell strainer. The cell suspension is then transferred to a tissue culture plate coated with appropriate extracellular matrix components (e.g., collagen, laminin, fibronectin).

Alternatively, explant specimens can be processed into small pieces, for example by mincing with a sterile scalpel. The tissue pieces are plated onto a suitable surface such as a gelatin sponge or a plastic tissue culture plate coated with appropriate extracellular matrix.

The plated cells are maintained at 37° C. in a humidified incubator with an atmosphere of 5% CO₂. During incubation, the media is exchanged about every 1 to 3 days and the cells are sub-cultured until a sufficient viable cell number is achieved for subsequent processing, which includes preparation for storage in liquid nitrogen; development of immortalized cell lines through the stable transfection of genes such as SV40, TERT, or other genes associated with senescence; isolation of mammary epithelial, myoepithelial, and stem/progenitor cell types by, for example, fluorescence-activated cell sorting; and/or introduction into a compartmentalizing tissue culture apparatus for the production and collection of milk for human consumption.

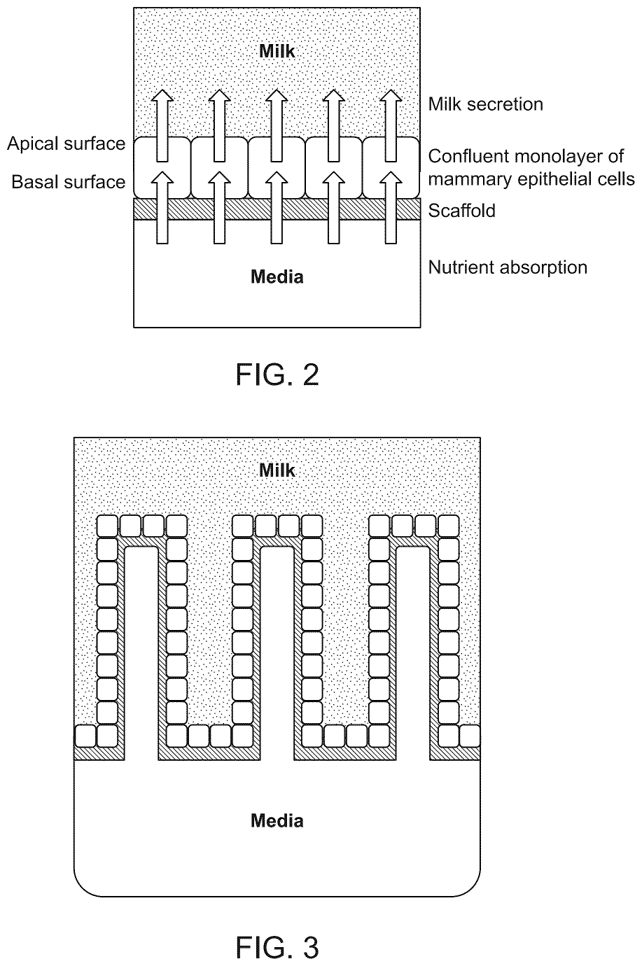

Cultivation of mammary epithelial cells for the production of milk. Milk for nutritional use is produced by mammary epithelial cells isolated as described above and cultured in a format that supports compartmentalized secretion such that separation between the nutrient medium and the product is maintained. The system relies on the ability of mammary epithelial cells to establish a continuous monolayer with appropriate apical-basal polarity when seeded onto an appropriate scaffold positioned at the interface between the apical compartment, into which milk is secreted, and the basal compartment, through which nutrient media is provided (see, e.g., FIG. 2). Transwell® filters placed in tissue culture plates, as well as bioreactors based on hollow fiber or microstructured scaffolds, for example, are used to support these characteristics.

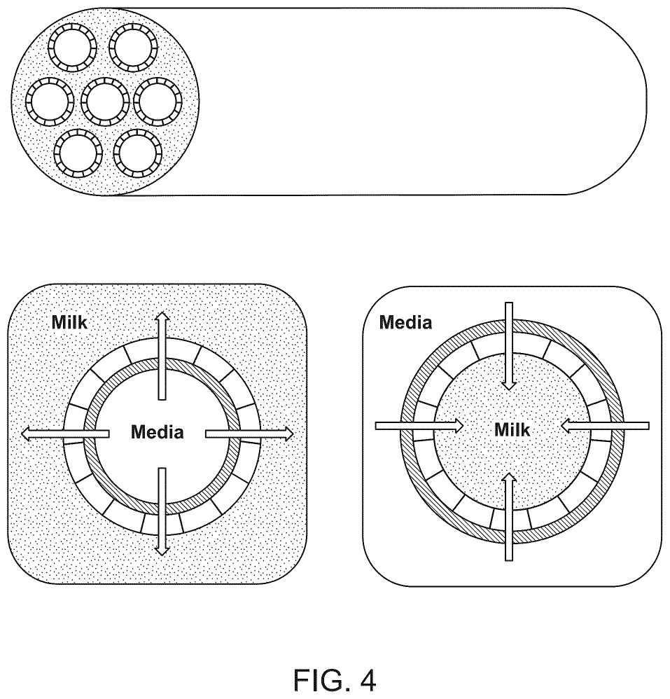

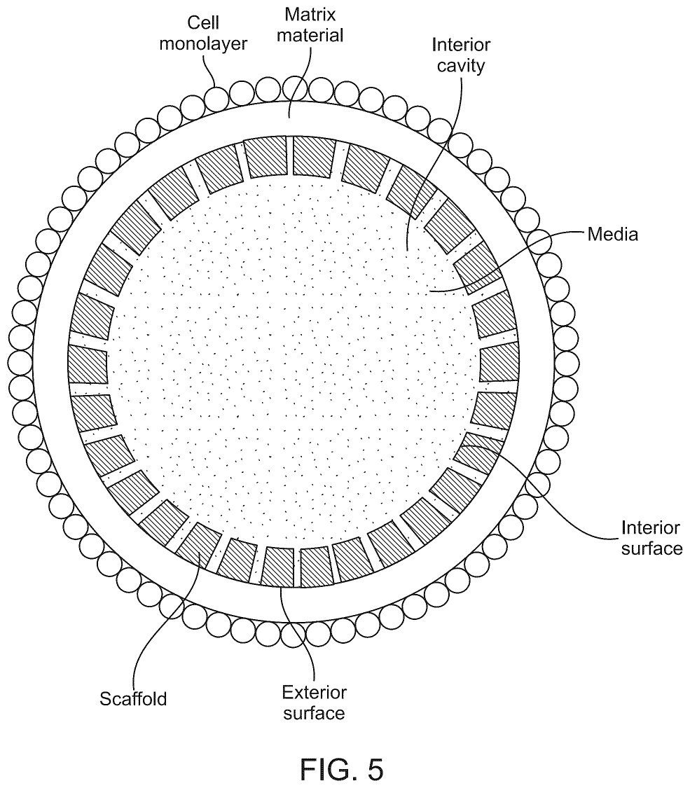

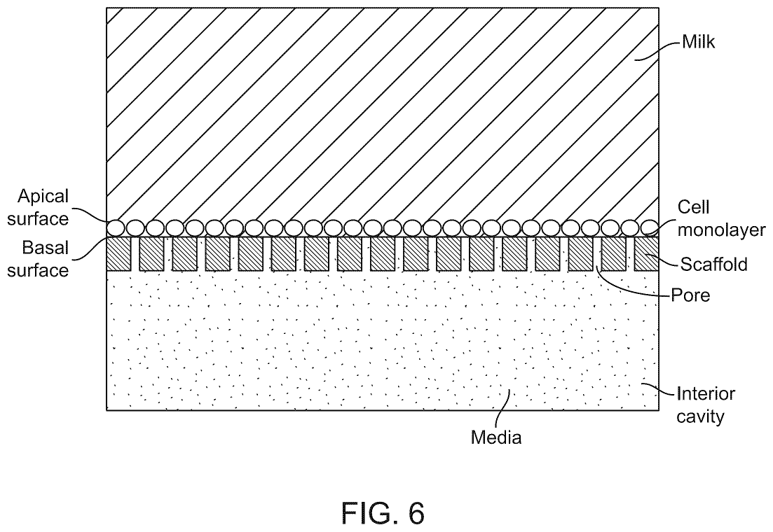

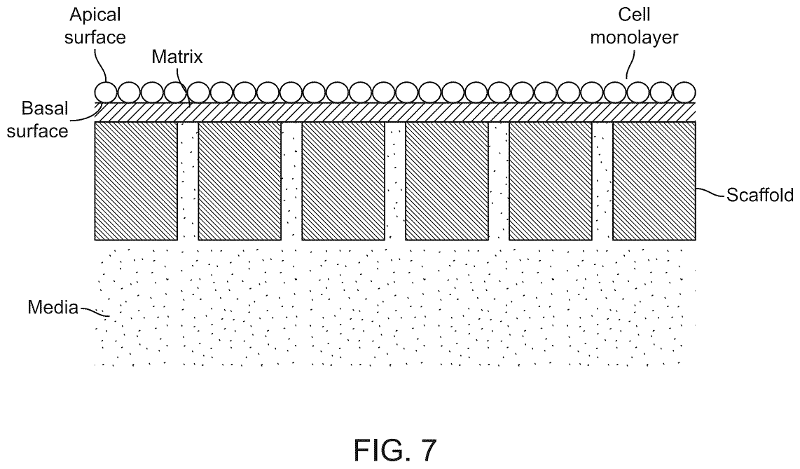

Following the isolation and expansion of mammary epithelial cells, the cells are suspended in a chemically defined nutrient medium composed of food-grade components and inoculated into a culture apparatus that has been pre-coated with a mixture of extracellular matrix proteins, such as collagen, laminin, and/or fibronectin. The cell culture apparatus is any design that allows for the compartmentalized absorption of nutrients and secretion of product from a polarized, confluent, epithelial monolayer. Examples include hollow fiber and microstructured scaffold bioreactors (see, e.g., FIGS. 3 and 4, respectively). Alternatives include other methods of 3-dimensional tissue culture, such as the preparation of decellularized mammary gland as a scaffold, repopulated with stem cells to produce a functional organ in vitro, or collection of milk from the lumen of mammary epithelial cell organoids or “mammospheres” grown either in a hydrogel matrix or in suspension.

The apparatus includes sealed housing that maintains a temperature of about 37° C. in a humidified atmosphere of about 5% CO₂. Glucose uptake is monitored to evaluate the growth of the culture as the cells proliferate within the bioreactor. Stabilization of glucose consumption indicates that the cells have reached a confluent, contact-inhibited state. The integrity of the monolayer is ensured using transepithelial electrical resistance. Sensors monitor concentrations of dissolved O₂ and CO₂ in the media at multiple locations. A computerized pump circulates media through the bioreactor at a rate that balances the delivery of nutrients with the removal of metabolic waste such as ammonia and lactate. Media can be recycled through the system after removal of waste using Lactate Supplementation and Adaptation technology (Freund et al. 2018 Int J Mol Sci. 19(2)) or by passing through a chamber of packed zeolite.

Stimulation of milk production. In vivo and in cultured mammary epithelial cells, the production and secretion of milk is stimulated by prolactin. In culture, prolactin can be supplied exogenously in the nutrient media at concentrations approximating those observed in the body during lactation, e.g., about 20 ng/mL to about 200 ng/mL. Purified prolactin can be obtained commercially; however, alternative methods of providing prolactin or stimulating lactation are employed, including expression and purification of the recombinant protein from microbial or mammalian cell cultures. Alternatively, conditioned media prepared by culturing cells that express and secrete prolactin can be applied to mammary epithelial cell cultures to stimulate lactation. Bioreactors can be set up in series such that media passing through a culture of cells expressing prolactin or other key media supplements is conditioned prior to exposure to mammary cells grown in a compartmentalizing culture apparatus as described.

Other approaches to upregulate milk production and/or spare the use of exogenous prolactin include molecular manipulation of the signaling pathways that are regulated by binding of prolactin to its receptor on the surface of mammary epithelial cells, such as the following: (a) expression of constructs targeting the posttranslational modification of prolactin; (b) expression of alternative isotypes of the prolactin receptor; (c) expression of a chimeric prolactin receptor in which the extracellular domain is exchanged with the binding site for a different ligand; (d) introduction of a gene encoding a constitutively or conditionally active prolactin receptor or modified versions of its downstream effectors such as STAT5 or Akt; (c) knockout or modification of the PER2 circadian gene; and/or (f) molecular approaches aimed at increasing the rate of nutrient uptake at the basal surface of the mammary epithelial monolayer.

Collection of milk. Secreted milk is collected continuously or at intervals through, for example, a port installed in the apical compartment of the culture apparatus. A vacuum is applied to the port to facilitate collection and also contributes to the stimulation of further production. The collected milk is packaged into sterile containers and sealed for distribution, frozen or lyophilized for storage, or processed for the extraction of specific components.

The present invention provides mammary epithelial cell cultures for the production of milk for nutritional use. In addition to human breast milk, this method may be used to produce milk from other mammalian species, for example, for human consumption or veterinary use. Because it has not been previously possible to produce milk outside the body, this technology may result in novel commercial opportunities, in addition to providing an alternative mode of production for existing products. The social and economic effects of the commercial development of this technology are broad and far reaching. Production of human breast milk from cultured cells may provide a means to address infant malnutrition in food-scarce communities, provide essential nutrients to premature infants who are unable to breastfeed, and offer mothers a new option for feeding their babies that provides optimal nutrition with the convenience of infant formula. Production of cow or goat milk provides an opportunity to reduce the environmental, social, and animal welfare effects of animal agriculture. The process described here addresses an important gap in the emerging field of cellular agriculture and introduces an opportunity to dramatically update the human food supply without compromising our biological and cultural attachment to the most fundamental of our nutrition sources.

The foregoing examples are illustrative of the present invention, and are not to be construed as limiting thereof. Although the invention has been described in detail with reference to preferred embodiments, variations and modifications exist within the scope and spirit of the invention as described and defined in the following claims.

What is claimed is:

1. An isolated milk product comprising at least one milk component secreted from cultured mammary cells; wherein the isolated milk product does not comprise cell culture medium, an environmental contaminant or a food allergen.

2. The isolated milk product of claim 1, wherein the isolated milk product comprises at least one milk protein secreted from the cultured mammary cells.

3. The isolated milk product of claim 1, produced by a method comprising:
a. culturing a live cell construct in a bioreactor under conditions which produce a milk product,
said live cell construct comprising:
i. a three-dimensional scaffold having an exterior surface, an interior surface defining an interior cavity, and a plurality of pores extending from the interior surface to the exterior surface;
ii. a matrix material disposed on the exterior surface of the three-dimensional scaffold;
iii. a culture medium disposed within the interior cavity and in fluidic contact with the internal surface; and
iv. a confluent monolayer of polarized mammary cells disposed on the matrix material, wherein the mammary cells are selected from the group consisting of: live primary mammary epithelial cells, live mammary myoepithelial cells, live immortalized mammary epithelial cells, and live immortalized mammary myoepithelial cells, and wherein the polarized mammary cells comprise an apical surface from which the milk product is secreted and a basal surface;
said bioreactor comprising an apical compartment that is in fluidic contact with the apical surface of the mammary cells, is substantially isolated from the interior cavity of the live cell construct, and is substantially free of cell culture medium; and
b. isolating the milk product secreted into the apical compartment from the apical surface of the mammary cells.

4. The isolated milk product of claim 1, wherein the environmental contaminant is a persistent organic pollutant (POP).

5. The isolated milk product of claim 1, wherein the POP is selected from polychlorinated dibenzo-p-dioxin (PCDD), polychlorinated dibenzofuran (PCDF), polychlorinated biphenyl (PCB), and DDT.

6. The isolated milk product of claim 1, wherein the environmental contaminant is a heavy metal.

7. The isolated milk product of claim 1, wherein the food allergen is selected from ovomucoid, ovalbumin, conalbumin, arachin 6, arachin 3, conarachin, Arah1, and Arah2.

8. The isolated milk product of claim 1, wherein the isolated milk product is sterile.

9. The isolated milk product of claim 1, wherein the isolate milk product is frozen or lyophilized.

10. The isolated milk product of claim 1, wherein the isolate milk product is in a canister, jar, bottle, bag, box or pouch.
